(19) United States
(12) Patent Application Publication
(10) Pub. No.: US 2026/0325169 A1
(43) Pub. Date: Oct. 22, 2026

(54) TROCARS WITH SEALING ASSEMBLIES FOR MINIMALLY INVASIVE SURGICAL APPLICATIONS

(71) Applicant: Vicarious Surgical Inc., Waltham, MA (US)

(72) Inventors: Allison Marie Stauffer, Cambridge, MA (US); Maeve Devlin, East Boston, MA (US); Rolando Rodarte, Cambridge, MA (US); Vinit Patel, Waltham, MA (US); Thomas Grosskopf, Hingham, MA (US); Susan Su, Waltham, MA (US); Jesse Dlugos, Woodbridge, CT (US); Alan Bachman, Woodbridge, CT (US); Robert Percy, Woodbridge, CT (US); Alexander Dixit, Somerville, MA (US); Jason Philip Cope, Somerville, MA (US)

(21) Appl. No.: 19/114,730

(22) PCT Filed: Sep. 29, 2023
(86) PCT No.: PCT/US2023/034158

§ 371 Date: Mar. 24, 2025

Related U.S. Application Data
(60) Provisional application No. 63/412,359, filed on Sep. 30, 2022.

(51) Int. Cl. A61B 17/34; A61B 34/30

(52) U.S. Cl. CPC A61B 17/3423; A61B 17/3474; A61B 34/30; A61B 2017/3419; A61B 2034/302; A61B 2217/005; A61B 2218/008

(57) ABSTRACT

Trocars including a cannula and a seal assembly are disclosed herein. The seal assembly may include a first layer of a flexible material and a second layer of a flexible material. The first layer may include a plurality of first layer individual seals and the second layer may include a plurality of individual seals each aligned with a corresponding one of the first layer individual seals. The seal assembly may include a seal support structure supported, at least in part, by the first layer. The seal support structure defines, at least in part, a plurality of channels each corresponding to a first layer individual seal and a second layer individual seal. The seal assembly may enable sealing and maintaining insufflation before, during and after insertion of multiple different instruments into the trocar.

### RELATED APPLICATION

This application claims the benefit of the filing date, under 35 U.S.C. § 119(e), of U.S. Provisional Application No. 63/412,359, filed on Sep. 30, 2022, the entire contents of which are incorporated herein by reference.

### BACKGROUND

Trocars are used in endoscopic surgical procedures to provide an access channel for cameras and tools to enter the surgical site. Most conventional trocars primarily accommodate one surgical instrument per incision. Because multiple tools are required to perform most endoscopic surgeries, a surgeon must make multiple incisions employing multiple different trocars to successfully complete a procedure. This process is time consuming, takes longer to heal, and introduces more potential points of infection in the patient.

Vital to the safety and efficacy of a trocar for an endoscopic procedure is the seal. Many conventional trocar systems have single layer seals. Trocars with single layer seals cannot seal the cannula adequately to maintain insufflation when no instruments are inserted while still allowing for easy passage of materials and surgical instruments. Further, conventional single layer seal trocar designs do not enable straight arm insertion of two robotic arms and a camera while maintaining a sufficient seal to maintain insufflation throughout the entire insertion and extraction process. Rather, a large incision is made to accommodate arm and camera insertion at varied angles. This time-consuming process requires a high degree of accuracy with regard to trocar positioning and requires a relatively large incision.

### SUMMARY OF THE DISCLOSURE

The present disclosure provides trocars and methods of using a trocar for surgical applications and methods of manufacturing a trocar. For example, the present disclosure provides a trocar for use in an endoscopic surgical procedure employing at least one endoscopic instrument or endoscopic camera. In various embodiments, the trocar includes a cannula having a central insertion axis and a proximal end defining a proximal opening. The trocar further includes a sealing assembly configured to seal the proximal opening of the cannula. The sealing assembly includes a seal support structure and at least a first layer and a second layer.

In various embodiments, the seal support structure defines, at least in part, a plurality of channels. The first layer covers the proximal opening of the cannula. In various embodiments, the first layer has a central portion and comprises a first flexible material. The seal support structure is attached to, or integral with, the central portion of the first layer. The first layer further includes a plurality of first layer individual seals. Each first layer individual seal has an opening for insertion of an endoscopic tool or endoscopic instrument.

In various embodiments, the second layer comprises a second flexible material and includes a plurality of second layer individual seals. Each second layer individual seal has an opening for insertion of an endoscopic tool or endoscopic instrument. In various embodiments, each first layer individual seal is aligned both with a corresponding second layer individual seal and with a channel of the plurality of channels in the seal support structure. Further, each first layer individual seal is displaced in a proximal or a distal direction from the corresponding second layer individual seal.

In various embodiments, the sealing assembly is configured to enable at least part of the central portion of the first layer to translate radially with respect to the central insertion axis. In doing so, the sealing assembly is configured to move a first layer individual seal and a corresponding second layer individual seal radially closer to the central insertion axis of the cannula in use for alignment of an endoscopic instrument or tool with the central insertion axis of the cannula.

In various embodiments, the first layer of the trocar further comprises one or more concentric corrugations or pleats to enable radial translation of the central portion of the first layer.

In various embodiments, the first layer of the trocar further comprises a peripheral portion. The one or more concentric corrugations or pleats are disposed in the peripheral portion of the first layer and encircle the central portion.

In various embodiments, each second layer individual seal comprises a slit formed in the second flexible material.

In various embodiments, each first layer individual seal comprises a universal seal.

In various embodiments, each first layer individual seal comprises a cross-slit valve or a duckbill valve.

In various embodiments, the first layer further comprises a first sheet having the first flexible material and a second sheet comprising the first flexible material. Each first layer individual seal includes a first slit in the first sheet and a second slit in second sheet, where an orientation of the first slit is different than an orientation of the second slit, thus forming a cross-slit valve.

In various embodiments, the first layer further comprises a seal sheet made from the first flexible material. In some embodiments, a peripheral portion of the first layer and each first layer individual seal is integral with the seal sheet.

In various embodiments, the second layer comprises a second seal sheet made from the second material. In some embodiments, each second layer individual seal is integral with the second seal sheet.

In various embodiments, the first layer individual seals are separable from the first layer and the second layer individual seals are separable from the second layer.

In various embodiments, the seal support structure comprises a brace plate disposed proximal relative to the first layer and the second layer with respect to the central insertion axis. In some embodiments, the brace plate is disposed distal relative to the first layer and the second layer with respect to the central insertion axis. In other embodiments, the brace plate is disposed between the first layer and the second layer.

In various embodiments, the trocar further comprises a brace plate support layer. In this embodiment, the brace plate is attached to and supported by the brace plate support layer.

In various embodiments, the brace plate support layer has central portion and a peripheral portion. The brace plate support layer includes one or more concentric corrugations or pleats disposed in the peripheral portion and encircling the central portion.

In various embodiments, the seal support structure comprises or further comprises one or more multi-lobed plates. In some embodiments, each lobe defines an opening corresponding to an aperture in the plurality of apertures.

In various embodiments, the seal support structure of the trocar comprises a plurality of plates.

In various embodiments, the first layer of the trocar is disposed between a first plate of the plurality of plates and a second plate of the plurality of plates.

In various embodiments, the second layer is disposed between the second of the plurality of plates and a third of the plurality of plates.

In various embodiments, the first layer and the second layer are held between the first plate of the plurality of plates and the second plate of the plurality of plates.

In various embodiments, the seal support structure comprises a first plate including a plurality of apertures, each defining a portion of a corresponding channel in the plurality of channels.

In various embodiments, the seal support structure further comprises a second plate, the second plate including at least one aperture connecting with the plurality of channels.

In various embodiments, the seal support structure is supported, at least in part, by the first layer.

In various embodiments, the second layer covers the proximal opening of the cannula. In such embodiments, the second layer supports, or supports in part, the seal support structure.

In various embodiments, the second layer has a central portion and a peripheral portion. In such embodiments, the second layer includes one or more concentric corrugations or pleats formed in the peripheral portion surrounding the central portion.

In various embodiments, the second layer is disposed proximal relative to the first layer with respect to the central insertion axis.

In various embodiments, the second layer is disposed distal relative to the first layer with respect to the central insertion axis.

The present disclosure provides another embodiment of a trocar for use in an endoscopic surgical procedure employing at least one endoscopic instrument or endoscopic camera. For example, the present disclosure provides a trocar having a cannula having a central insertion axis and a proximal end defining a proximal opening. The trocar further includes a sealing assembly configured to seal the proximal end of the cannula. In various embodiments, the first layer of the sealing assembly covers the proximal opening of the cannula. The first layer of the sealing assembly has a central portion and comprises a first flexible material. The first layer includes a plurality of first layer individual seals in the central portion. Each first layer individual seal has an opening for insertion of an endoscopic instrument or an endoscopic camera.

The sealing assembly of the trocar further includes a second layer comprising a second flexible material. The second layer further includes a plurality of second layer individual seals. Each second layer individual seal has an opening for insertion of an endoscopic tool or endoscopic instrument. Further, each first layer individual seal is aligned with a corresponding second layer individual seal. Each first layer individual seal is displaced in a proximal or distal direction with respect to the central insertion axis from the corresponding second layer individual seal.

Additionally, the sealing assembly is configured to enable at least part of the central portion of the first layer to translate radially with respect to the central insertion axis. In doing so, the central portion moves a first layer individual seal and a corresponding second layer individual seal radially closer to the central insertion axis of the cannula in use for alignment of an endoscopic instrument or tool with the central insertion axis of the cannula.

In various embodiments, the first layer, the second layer, or both layer of the trocar define, at least in part, a plurality of lumens.

In various embodiments, the trocar further comprising a seal support structure having a central aperture. The central aperture corresponds to the central portion of the first layer in some embodiments. Further, the seal support structure is affixed, either directly or indirectly the first layer. Additionally, the seal support structure is also supported, at least in part, by the first layer.

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. These and other features and advantages of the present invention will be more fully understood by reference to the following detailed description in conjunction with the accompanying figures in which like reference numerals refer to like elements throughout the different views.

To assist those of skill in the art in making and using the trocar of the present disclosure, reference is made to the accompanying figures, wherein:

FIG. 1 schematically depicts a surgical robotic system in accordance with some embodiments.

FIG. 2A is a perspective view of a patient cart including a robotic support system coupled to a robotic subsystem of the surgical robotic system in accordance with some embodiments.

FIG. 2B is a perspective view of an example operator console of a surgical robotic system of the present disclosure in accordance with some embodiments.

FIG. 3A schematically depicts a side view of a surgical robotic system performing a surgery within an internal cavity of a subject in accordance with some embodiments.

FIG. 3B schematically depicts a top view of the surgical robotic system performing the surgery within the internal cavity of the subject of FIG. 3A in accordance with some embodiments.

FIG. 4A is a perspective view of a single robotic arm subsystem or subassembly in accordance with some embodiments.

FIG. 4B is a perspective side view of a single robotic arm of the single robotic arm subsystem of FIG. 4A in accordance with some embodiments.

FIG. 5 is a perspective front view of a camera assembly and a robotic arm assembly in accordance with some embodiments.

FIG. 6 is a perspective view of a trocar in accordance with some embodiments of the present disclosure.

FIG. 7 is a section view of a proximal portion of the trocar of FIG. 6 in accordance with embodiments of the present disclosure.

FIG. 8 depicts a section view of a trocar in accordance with some embodiments of the present disclosure.

FIG. 9 is a perspective view of a proximal end of the trocar of FIG. 8.

FIG. 10 is a perspective view of the proximal end of the trocar of FIG. 8 with an outer cannula cap and mounting adaptor omitted for illustrative purposes.

FIG. 11A is an exploded perspective view of a seal assembly of the trocar of FIG. 8.

FIG. 11B is an exploded perspective view of a seal assembly of a trocar in accordance with some embodiments.

FIG. 11C includes images of leaflets insertable into a trocar in accordance with some embodiments.

FIG. 12 is a section view of a proximal end of a trocar in accordance with another embodiment.

FIG. 13 is a proximal end view of a mounting bracket and an outer cannula cap attached to the trocar of FIG. 12.

FIG. 14 include an image of an individual first layer valve created by forming a slit in each of two flexible sheets in accordance with an embodiment.

FIG. 15 is a perspective view of a trocar including a brace plate attached to a flexible layer in accordance with some embodiments.

FIG. 16 is a perspective view of the brace plate attached to the flexible layer of FIG. 16.

FIG. 17 is a perspective view of a trocar including bellow plate in accordance with some embodiments.

FIG. 18A is a perspective view of a trocar cap including sliding support clips being used with a trocar in accordance with some embodiments.

FIG. 18B is a top view of the trocar cap of FIG. 18A.

FIG. 18C is an image of a top view of a prototype trocar cap including sliding support clips attached to a trocar in accordance with some embodiments.

FIG. 19A is a side view of a cannula including a smoke evacuation lumen and a smoke evacuation port or connection in accordance with some embodiments.

FIG. 19B is a side cross-section view of the cannula of FIG. 19A.

FIG. 19C is a side cross-section view of a proximal portion of the cannula of FIG. 19A.

FIG. 20A is a perspective view of a trocar including a trocar funnel with individual seals in accordance with some embodiments.

FIG. 20B is an exploded perspective view of the trocar of FIG. 20A.

FIG. 20C is a perspective view of a cannula body of the trocar of FIG. 20A.

FIG. 20D is a cross-sectional view of the trocar funnel of the trocar of FIG. 20A.

FIG. 21A is an exploded schematic view of a trocar including an integrated flexible seal piece in accordance with some embodiments.

FIG. 21B is a schematic side view of the trocar of FIG. 21A identifying a docketing interface.

FIG. 21C is a schematic perspective view of the cannula body of the trocar of FIG. 21A.

FIG. 21D is a schematic perspective view of the seal piece of the trocar of FIG. 21A.

FIG. 21E is a schematic cross-section view of an individual seal of the seal piece of FIG. 21A.

FIG. 21F is a schematic perspective view of a guide cap of the trocar of FIG. 21A.

FIG. 22A is a schematic side cross-section view of a rolling seal being used to seal an instrument being inserted into a trocar in accordance with some embodiments.

FIG. 22B is a schematic side cross-section view of the rolling seal, instrument, and trocar of FIG. 22A after the instrument has been further inserted and the seal is fully within the trocar.

FIG. 22C is a schematic side cross-section view of multiple rolling seals and instruments being inserted into a trocar in accordance with some embodiments.

FIG. 23A is a front perspective view of a cork coupleable to a camera support tube insertable into a trocar in accordance with some embodiments.

FIG. 23B is a side view of the cork of FIG. 23A.

FIG. 23C is a rear perspective view of the cork of FIG. 23A.

FIG. 23D is another side view of the cork of FIG. 23A.

FIG. 24 is a perspective view of the cork of FIG. 23A situated around a support tube in accordance with some embodiments.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It may be understood that various alternatives to the embodiments of the invention described herein may be employed.

As used in the specification and claims, the singular form “a,” “an,” and “the” include plural references unless the context clearly dictates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Furthermore, the use of the term “including” as well as other forms, such as “includes” and “included,” is not limiting.

In this application, the use of the singular includes the plural unless specifically stated otherwise. In this application, the use of “or” means “and/or” unless stated otherwise.

Further, as used herein, the terms “or” and “and/or” include any and all combinations of one or more of the associated listed items.

As used herein with respect to insertion into and through a trocar or cannula, the terms “endoscopic camera” and “camera” should be interpreted to include a camera unit, a camera assembly, a camera body, a camera module, or other structure, device or system including at least one imaging device that is configured to be entirely inserted into an internal cavity of a body via a trocar or cannula, or the portion of the aforementioned that is configured to be entirely inserted into an internal cavity of a body via the trocar or cannula. As used herein with respect to insertion into and through a trocar or cannula, an endoscopic camera or a camera can include multiple imaging devices, that can include, but are not limited to, imaging devices for light in the visible spectrum, for light in the nonvisible spectrum, or both.

The term support shaft or support tube as used herein with respect to insertion into a trocar or a cannula refers to a structure that extends through a trocar or cannula and supports a tool, instrument, robotic arm, or camera that is entirely inserted into an internal body cavity. Disclosure herein regarding a support tube should be understood to be applicable to a support shaft.

As used herein, “endoscopic instrument” and its plural, “endoscopic instruments,” can refer to any variety of instruments and tools used by a surgeon during endoscopic surgery that are inserted through trocar into a surgical site or an internal body cavity. For example, “endoscopic instruments” can include, but are not limited to, endoscopic cameras, needles, needle holders, needle drivers, graspers, hooks, blades, forceps, loops, shears, scissors, injection tools, suction devices, wound closure devices, staplers, ligation devices, electrodes and electrocautery tools.

As used herein, “proximal” refers to relatively closer to the surgeon and further from an interior cavity of a patient in use, and “distal” refers to relatively further away from the surgeon and closer to or in an interior cavity of a patient in use.

The section headings used herein are for organizational purposes only and are not to be construed as limiting the subject matter described.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

When an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

Although some exemplary embodiments may be described herein or in documents incorporated by reference as employing a plurality of units to perform exemplary processes, it is understood that exemplary processes may also be performed by one or a plurality of modules. Additionally, it is understood that the term controller/control unit may refer to a hardware device that includes a memory and a processor and is specifically programmed to execute the processes described herein in accordance with some embodiments. In some embodiments, the memory is configured to store the modules and the processor is specifically configured to execute said modules to perform one or more processes which are described further below. In some embodiments, multiple different controllers or control units or multiple different types of controllers or control units may be employed in performing one or more processes. In some embodiments, different controllers or control units may be implemented in different portions of a surgical robotic system.

While exemplary embodiments of a trocar are described herein, one of ordinary skill in the art will recognize that embodiments are not limited to the illustrative embodiments. One of ordinary skill in the art will further recognize that trocars of the present disclosure can be used as a surgical aid for a variety of indications, patients, and procedures. In addition, components of the exemplary trocar and methods of using said trocar are not limited to the illustrative embodiments described below.

Embodiments of the present disclosure provide trocars and methods of using a trocar for surgical applications (e.g., endoscopic surgical applications). In some embodiments, the trocar is configured for use in endoscopic surgical procedures in which at least one endoscopic instrument or endoscopic camera is inserted through the trocar. In some embodiments, the trocar is configured for use in endoscopic surgical procedures in which at least two endoscopic instruments are inserted through the trocar. In some embodiments, the trocar is configured for use in an endoscopic surgical procedure in which at least one endoscopic instrument and an endoscopic camera are inserted through the trocar. In some embodiments, the trocar is configured for use with a surgical robotic system including at least one robotic arm and at least one camera to be operated through the same trocar. In some embodiments, the trocar is configured for use with a surgical robotic system including at least two robotic arms and at least one camera to be operated through the same trocar.

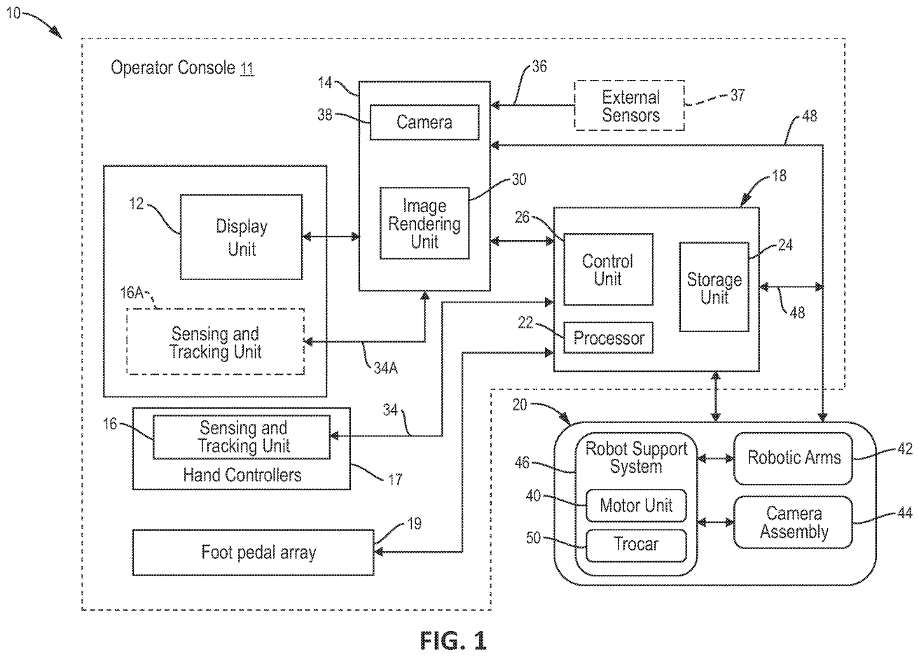

Prior to providing specific description of embodiments of trocars, cannulas, and seals with respect to FIGS. 6-22C, a surgical robotic system with which or in which embodiments of the trocar can be employed is described below with respect to FIGS. 1-5. One of ordinary skill in the art in view of the present disclosure will appreciate that embodiments described herein are not limited to use with the surgical robotic system described with respect to FIGS. 1-5, but can be employed with other surgical robotic systems and in surgical procedures that do not employ a surgical robotic system, or that only employ some robotic components.

### 1.

### Surgical Robotic System

Some embodiments may be employed with a surgical robotic system. A system for robotic surgery may include a robotic subsystem. The robotic subsystem includes at least portion that can be inserted into a patient via a trocar through a single incision point or site. The portion inserted into the patient via a trocar is small enough to be deployed in vivo at the surgical site and is sufficiently maneuverable when inserted to be able to move within the body to perform various surgical procedures at multiple different points or sites. The portion inserted into the body that performs functional tasks may be referred to as a surgical robotic module or a surgical robotic unit. The surgical robotic module or surgical robotic unit can include multiple different submodules, subunits, or portions that may be inserted into the trocar separately. The surgical robotic module can include multiple separate robotic arms that are deployable within the patient along different or separate axes. Further, a surgical camera assembly can also be deployed along a separate axis. Thus, the surgical robotic module employs multiple different components, such as a pair of robotic arms and a surgical or robotic camera assembly, each of which are deployable along different axes and are separately manipulatable, maneuverable, and movable.

The robotic arms and the camera assembly that are disposable along separate and manipulatable axes is referred to herein as the Split Arm (SA) architecture. The SA architecture is designed to simplify and increase efficiency of the insertion of robotic surgical instruments through a single trocar at a single insertion site, while concomitantly assisting with deployment of the surgical instruments into a surgical ready state as well as the subsequent removal of the surgical instruments through the trocar. By way of example, a surgical instrument can be inserted through the trocar to access and perform an operation in vivo in the abdominal cavity of a patient. In some embodiments, various surgical instruments may be used or employed, including but not limited to robotic surgical instruments, as well as other surgical instruments known in the art.

The systems, devices, and methods disclosed herein can be incorporated into and/or used with a robotic surgical device and associated system disclosed for example in U.S. Pat. No. 10,285,765 and in PCT patent application Serial No. PCT/US2020/39203, and/or with the camera assembly and system disclosed in United States Publication No. 2019/0076199, and/or the systems and methods of exchanging surgical tools in an implantable surgical robotic system disclosed in PCT patent application Serial No. PCT/US2021/058820, where the content and teachings of all of the foregoing patents, patent applications and publications are incorporated herein by reference herein in their entirety. The surgical robotic module can be part of a robotic subsystem of a surgical robotic system. The surgical robotic system may include a surgeon workstation or operator console that includes appropriate sensors and displays, and a robot support system (RSS) for interacting with and supporting the robotic subsystem in some embodiments. The robotic subsystem includes a motor module and the surgical robotic module that includes one or more robotic arms and one or more camera assemblies in some embodiments. The robotic arms and camera assembly can form part of a single support axis robotic system, can form part of the split arm (SA) architecture robotic system, or can have another arrangement. The robot support system can provide multiple degrees of freedom such that the surgical robotic module can be maneuvered within the patient into a single position or multiple different positions. In one embodiment, the robot support system can be directly mounted to a surgical table or to the floor or ceiling within an operating room. In another embodiment, the mounting is achieved by various fastening means, including but not limited to, clamps, screws, or a combination thereof. In other embodiments, the structure may be free standing. The robot support system can mount a motor assembly that is coupled to the surgical robotic module, which includes the robotic arms and the camera assembly. The motor assembly can include gears, motors, drivetrains, electronics, and the like, for powering the components of the surgical robotic unit.

The robotic arms and the camera assembly are capable of multiple degrees of freedom of movement. According to some embodiments, when the robotic arms and the camera assembly are inserted into a patient through the trocar, they are capable of movement in at least the axial, yaw, pitch, and roll directions. The robotic arms are designed to incorporate and employ a multi-degree of freedom of movement robotic arm with an end effector mounted at a distal end thereof that corresponds to a wrist area or joint of the user. In other embodiments, the working end (e.g., the end effector end) of the robotic arm is designed to incorporate and use or employ other robotic surgical instruments, such as for example the surgical instruments set forth in U.S. Publ. No. 2018/0221102, the entire contents of which are herein incorporated by reference.

Turning to the drawings, FIG. 1 is a schematic illustration of an example surgical robotic system 10 in which aspects of the present disclosure can be employed in accordance with some embodiments. The surgical robotic system 10 includes an operator console 11 and a robotic subsystem 20 in accordance with some embodiments.

The operator console 11 includes a display device or unit 12, an image computing unit 14, which may be a three-dimensional (3-D) computing unit, hand controllers 17 having a sensing and tracking unit 16, and a computing unit 18. Additionally, the operator console 11 may include foot pedal array 19.

The display unit 12 may be any selected type of display for displaying information, images or video generated by the image computing unit 14, the computing unit 18, and/or the robotic subsystem 20. The display unit 12 can include or form part of, for example, a head-mounted display (HMD), an augmented reality (AR) display (e.g., an AR display, or AR glasses in combination with a screen or display), a screen or a display, a two-dimensional (2D) screen or display, a three-dimensional (3D) screen or display, and the like. The display unit 12 can also include an optional sensing and tracking unit 16A. In some embodiments, the display unit 12 can include an image display for outputting an image from a camera assembly 44 (see FIG. 1) of the robotic subsystem 20.

In some embodiments, if the display unit 12 includes an HMD device, an AR device that senses head position, or another device that employs an associated sensing and tracking unit 16A, the HMD device or head tracking device generates tracking and position data 34A that is received and processed by image computing unit 14. In some embodiments, the HMD, AR device, or other head tracking device can provide an operator (e.g., a surgeon, a nurse or other suitable medical professional) with a display that is at least in part coupled or mounted to the head of the operator, lenses to allow a focused view of the display, and the sensing and tracking unit 16A to provide position and orientation tracking of the operator's head. The sensing and tracking unit 16A can include for example accelerometers, gyroscopes, magnetometers, motion processors, infrared tracking, eye tracking, computer vision, emission and sensing of alternating magnetic fields, and any other method of tracking at least one of position and orientation, or any combination thereof. In some embodiments, the HMD or AR device can provide image data from the camera assembly 44 to the right and left eyes of the operator. In some embodiments, in order to maintain a virtual reality experience for the operator, the sensing and tracking unit 16A, can track the position and orientation of the operator's head, generate tracking and position data 34A, and then relay the tracking and position data 34A to the image computing unit 14 and/or the computing unit 18 either directly or via the image computing unit 14.

The hand controllers 17 are configured to sense a movement of the operator's hands and/or arms to manipulate the surgical robotic system 10. The hand controllers 17 can include the sensing and tracking unit 16, circuity, and/or other hardware. The sensing and tracking unit 16 can include one or more sensors or detectors that sense movements of the operator's hands. In some embodiments, the one or more sensors or detectors that sense movements of the operator's hands are disposed in a pair of hand controllers that are grasped by or engaged by hands of the operator. In some embodiments, the one or more sensors or detectors that sense movements of the operator's hands are coupled to the hands and/or arms of the operator. For example, the sensors of the sensing and tracking unit 16 can be coupled to a region of the hand and/or the arm, such as the fingers, the wrist region, the elbow region, and/or the shoulder region. If the HMD is not used, then additional sensors can also be coupled to a head and/or neck region of the operator in some embodiments. If the operator employs the HMD, then the eyes, head and/or neck sensors and associated tracking technology can be built-in or employed within the HMD device, and hence form part of the optional sensor and tracking unit 16A as described above. In some embodiments, the sensing and tracking unit 16 can be external and coupled to the hand controllers 17 via electricity components and/or mounting hardware. In some embodiments, the optional sensor and tracking unit 16A may sense and track movement of one or more of an operator's head, of at least a portion of an operator's head, an operator's eyes or an operator's neck based, at least in part, on imaging of the operator in addition to or instead of by a sensor or sensors attached to the operator's body.

In some embodiments, the sensing and tracking unit 16 can employ sensors coupled to the torso of the operator or any other body part. In some embodiments, the sensing and tracking unit 16 can employ in addition to the sensors an Inertial Momentum Unit (IMU) having for example an accelerometer, gyroscope, magnetometer, and a motion processor. The addition of a magnetometer allows for reduction in sensor drift about a vertical axis. In some embodiments, the sensing and tracking unit 16 also include sensors placed in surgical material such as gloves, surgical scrubs, or a surgical gown. The sensors can be reusable or disposable.

In some embodiments, sensors can be disposed external of the operator, such as at fixed locations in a room, such as an operating room. The external sensors 37 can generate external data 36 that can be processed by the computing unit 18 and hence employed by the surgical robotic system 10.

The sensors generate position and/or orientation data indicative of the position and/or orientation of the operator's hands and/or arms. The sensing and tracking units 16 and/or 16A can be utilized to control movement (e.g., changing a position and/or an orientation) of the camera assembly 44 and robotic arms 42 of the robotic subsystem 20. The tracking and position data 34 generated by the sensing and tracking unit 16 can be conveyed to the computing unit 18 for processing by at least one processor 22.

The computing unit 18 can determine or calculate, from the tracking and position data 34 and 34A, the position and/or orientation of the operator's hands or arms, and in some embodiments of the operator's head as well, and convey the tracking and position data 34 and 34A to the robotic subsystem 20. The tracking and position data 34, 34A can be processed by the processor 22 and can be stored for example in the storage unit 24. The tracking and position data 34 and 34A can also be used by the control unit 26, which in response can generate control signals for controlling movement of the robotic arms 42 and/or the camera assembly 44. For example, the control unit 26 can change a position and/or an orientation of at least a portion of the camera assembly 44, of at least a portion of the robotic arms 42, or both. In some embodiments, the control unit 26 can also adjust the pan and tilt of the camera assembly 44 to follow the movement of the operator's head.

The robotic subsystem 20 can include a robot support system (RSS) 46 having a motor unit 40 and a trocar 50 or trocar mount, the robotic arms 42, and the camera assembly 44. The robotic arms 42 and the camera assembly may collectively be referred to as the surgical robotic module or surgical robotic unit. The robotic arms 42 and the camera assembly 44 can form part of a single support axis robot system, such as that disclosed and described in U.S. Pat. No. 10,285,765, or can form part of a split arm (SA) architecture robot system, such as that disclosed and described in PCT Patent Application No. PCT/US2020/039203, both of which are incorporated herein by reference in their entirety.

The robotic subsystem 20 can employ multiple different robotic arms that are deployable along different or separate axes. In some embodiments, the camera assembly 44, which can employ multiple different camera elements, can also be deployed along a common separate axis. Thus, the surgical robotic system 10 can employ multiple different components, such as a pair of separate robotic arms and the camera assembly 44, which are deployable along different axes. In some embodiments, the robotic arms 42 and the camera assembly 44 are separately manipulatable, maneuverable, and movable. The robotic subsystem 20, which includes the robotic arms 42 and the camera assembly 44, is disposable along separate manipulatable axes, and is referred to herein as an SA architecture. The SA architecture is designed to simplify and increase efficiency of the insertion of robotic surgical instruments through a single trocar at a single insertion point or site, while concomitantly assisting with deployment of the surgical instruments into a surgical ready state, as well as the subsequent removal of the surgical instruments through a trocar 50 as further described below.

The RSS 46 can include the motor unit 40 and the trocar 50 or a trocar mount. The RSS 46 can further include a support member that supports the motor unit 40 coupled to a distal end thereof. The motor unit 40 in turn can be coupled to the camera assembly 44 and to each of the robotic arms 42. The support member can be configured and controlled to move one or more components of the robotic subsystem 20 linearly, or in any other selected direction or orientation. In some embodiments, the RSS 46 can be free standing. In some embodiments, the RSS 46 can include the motor unit 40 that is coupled to the robotic subsystem 20 at one end and to an adjustable support member or element at an opposed end.

The motor unit 40 can receive the control signals generated by the control unit 26. The motor unit 40 can include gears, one or more motors, drivetrains, electronics, and the like, for powering and driving the robotic arms 42 and the cameras assembly 44 separately or together. The motor unit 40 can also provide mechanical power, electrical power, mechanical communication, and electrical communication to the robotic arms 42, the camera assembly 44, and/or other components of the RSS 46 and robotic subsystem 20. The motor unit 40 can be controlled by the computing unit 18. The motor unit 40 can thus generate signals for controlling one or more motors that, in turn, can control and drive the robotic arms 42, including for example the position and orientation of each articulating joint of each robotic arm, as well as the camera assembly 44. The motor unit 40 can further provide for a translational or linear degree of freedom that is first utilized to insert and remove each component of the robotic subsystem 20 through a trocar 50. The motor unit 40 can also be employed to adjust the inserted depth of each robotic arm 42 and the camera assembly 44 when inserted into the patient 100 through the trocar 50.

The trocar 50 is a medical device that can be made up of an obturator (which may be a metal or plastic sharpened or non-bladed tip), a cannula (essentially a hollow tube), and a seal in some embodiments. The trocar can be used to place at least a portion of the robotic subsystem 20 in an interior cavity of a subject (e.g., a patient) and can insert and/or withdraw gas and/or fluid from a body cavity. The robotic subsystem 20 can be inserted through the trocar to access and perform an operation in vivo in a body cavity of a patient. In some embodiments, the robotic subsystem 20 can be supported, at least in part, by the trocar 50 or a trocar mount with multiple degrees of freedom such that the robotic arms 42 and the camera assembly 44 can be maneuvered within the patient into a single position or multiple different positions. In some embodiments, the robotic arms 42 and camera assembly 44 can be moved with respect to the trocar 50 or a trocar mount with multiple different degrees of freedom such that the robotic arms 42 and the camera assembly 44 can be maneuvered within the patient into a single position or multiple different positions.

In some embodiments, the RSS 46 can further include an optional controller for processing input data from one or more of the system components (e.g., the display 12, the sensing and tracking unit 16, the robotic arms 42, the camera assembly 44, and the like), and for generating control signals in response thereto. The motor unit 40 can also include a storage element for storing data in some embodiments.

The robotic arms 42 can be controlled to follow the scaled-down movement or motion of the operator's arms and/or hands as sensed by the associated sensors in some embodiments and in some modes of operation. The robotic arms 42 include a first robotic arm including a first end effector at distal end of the first robotic arm, and a second robotic arm including a second end effector disposed at a distal end of the second robotic arm. In some embodiments, the robotic arms 42 can have portions or regions that can be associated with movements associated with the shoulder, elbow, and wrist joints as well as the fingers of the operator. For example, the robotic elbow joint can follow the position and orientation of the human elbow and the robotic wrist joint can follow the position and orientation of the human wrist. The robotic arms 42 can also have associated therewith end regions that can terminate in end-effectors that follow the movement of one or more fingers of the operator in some embodiments, such as for example the index finger as the user pinches together the index finger and thumb. In some embodiments, while the robotic arms 42 may follow movement of the arms of the operator in some modes of control, the robotic shoulders may be fixed in position in some modes of control. In some embodiments, the position and orientation of the torso of the operator are subtracted from the position and orientation of the operator's arms and/or hands. This subtraction allows the operator to move his or her torso without the robotic arms moving. Further disclosure control of movement of individual arms of a robotic arm assembly is provided in International Patent Application Publications WO 2022/094000 A1 and WO 2021/231402 A1, each of which is incorporated by reference herein in its entirety.

The camera assembly 44 is configured to provide the operator with image data 48, such as for example a live video feed of an operation or surgical site, as well as enable the operator to actuate and control the cameras forming part of the camera assembly 44. In some embodiments, the camera assembly 44 can include one or more cameras (e.g., a pair of cameras), the optical axes of which are axially spaced apart by a selected distance, known as the inter-camera distance, to provide a stereoscopic view or image of the surgical site. In some embodiments, the operator can control the movement of the cameras via movement of the hands via sensors coupled to the hands of the operator or via hand controllers grasped or held by hands of the operator, thus enabling the operator to obtain a desired view of an operation site in an intuitive and natural manner. In some embodiments, the operator can additionally control the movement of the camera via movement of the operator's head. The camera assembly 44 is movable in multiple directions, including for example in yaw, pitch and roll directions relative to a direction of view. In some embodiments, the components of the stereoscopic cameras can be configured to provide a user experience that feels natural and comfortable. In some embodiments, the interaxial distance between the cameras can be modified to adjust the depth of the operation site perceived by the operator.

The image or video data 48 generated by the camera assembly 44 can be displayed on the display unit 12. In embodiments in which the display unit 12 includes a HMD, the display can include the built-in sensing and tracking unit 16A that obtains raw orientation data for the yaw, pitch and roll directions of the HMD as well as positional data in Cartesian space (x, y, z) of the HMD. In some embodiments, positional and orientation data regarding an operator's head may be provided via a separate head-tracking unit. In some embodiments, the sensing and tracking unit 16A may be used to provide supplementary position and orientation tracking data of the display in lieu of or in addition to the built-in tracking system of the HMD. In some embodiments, no head tracking of the operator is used or employed. In some embodiments, images of the operator may be used by the sensing and tracking unit 16A for tracking at least a portion of the operator's head.

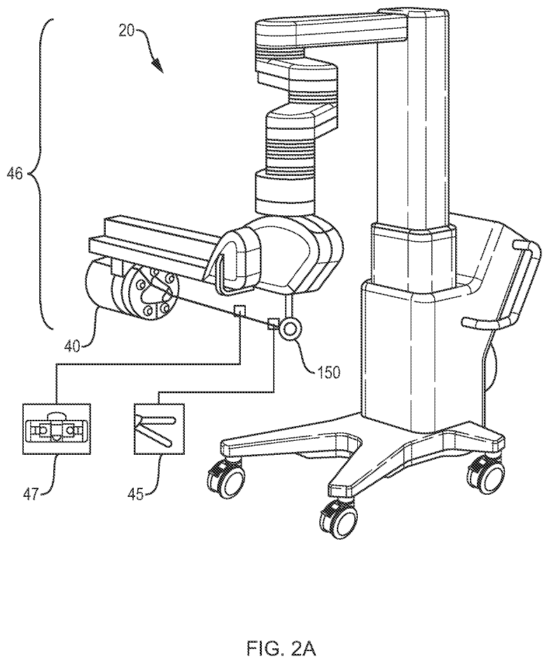

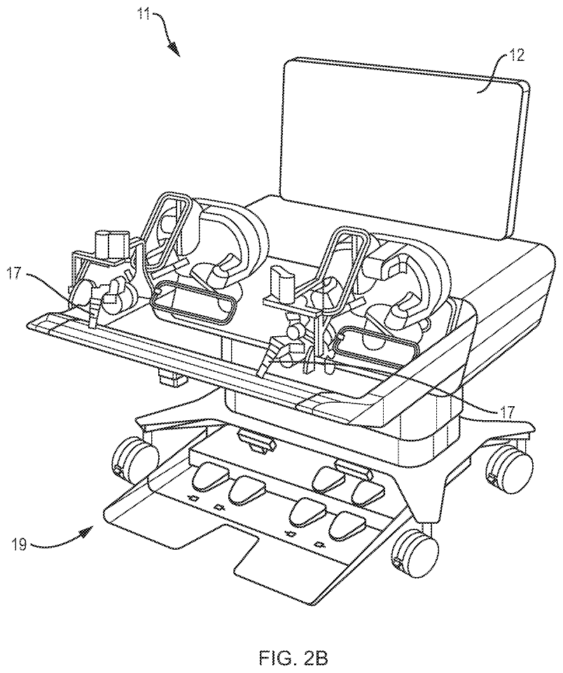

FIG. 2A depicts an example robotic assembly 20, which is also referred to herein as a robotic subsystem, of a surgical robotic system 10 incorporated into or mounted onto a mobile patient cart in accordance with some embodiments. In some embodiments, the robotic assembly 20 includes the RSS 46, which, in turn includes the motor unit 40, the robotic arm assembly 42 having end-effectors 45, the camera assembly 44 having one or more cameras 47, and may also include the trocar 50 or a trocar mount.

FIG. 2B depicts an example of an operator console 11 of the surgical robotic system 10 of the present disclosure in accordance with some embodiments. The operator console 11 includes a display unit 12, hand controllers 17, and also includes one or more additional controllers, such as a foot pedal array 19 for control of the robotic arms 42, for control of the camera assembly 44, and for control of other aspects of the system.

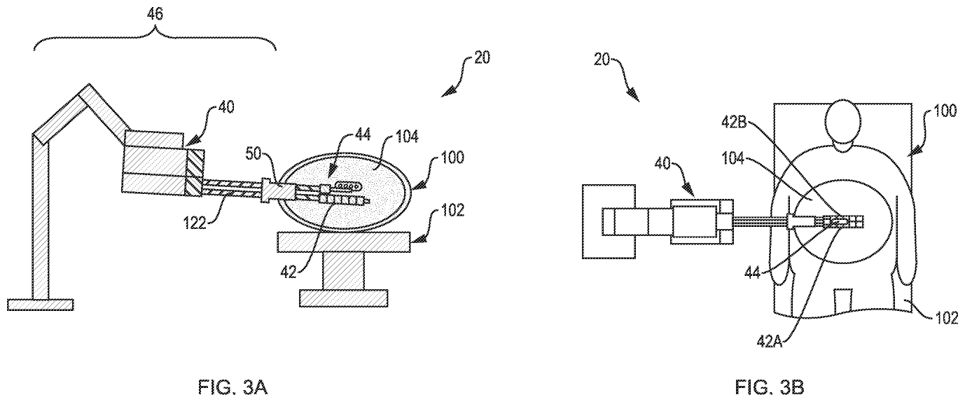

FIG. 3A schematically depicts a side view of the surgical robotic system 10 performing a surgery within an internal cavity 104 of a subject 100 in accordance with some embodiments and for some surgical procedures. FIG. 3B schematically depicts a top view of the surgical robotic system 10 performing the surgery within the internal cavity 104 of the subject 100. The subject 100 (e.g., a patient) is placed on an operation table 102 (e.g., a surgical table 102). In some embodiments, and for some surgical procedures, an incision is made in the patient 100 to gain access to the internal cavity 104. The trocar 50 is then inserted into the patient 100 at a selected location to provide access to the internal cavity 104 or operation site. The RSS 46 can then be maneuvered into position over the patient 100 and the trocar 50. In some embodiments, the RSS 46 includes a trocar mount that attaches to the trocar 50. The robotic assembly 20 can be coupled to the motor unit 40 and at least a portion of the robotic assembly can be inserted into the trocar 50 and hence into the internal cavity 104 of the patient 100. For example, the camera assembly 44 and the robotic arm assembly 42 can be inserted individually and sequentially into the patient 100 through the trocar 50. Although the camera assembly and the robotic arm assembly may include some portions that remain external to the subject's body in use, references to insertion of the robotic arm assembly 42 and/or the camera assembly into an internal cavity of a subject and disposing the robotic arm assembly 42 and/or the camera assembly 44 in the internal cavity of the subject are referring to the portions of the robotic arm assembly 42 and the camera assembly 44 that are intended to be in the internal cavity of the subject during use. The sequential insertion method has the advantage of supporting smaller trocars and thus smaller incisions can be made in the patient 100, thereby reducing the trauma experienced by the patient 100. In some embodiments, the camera assembly 44 and the robotic arm assembly 42 can be inserted in any order or in a specific order. In some embodiments, the camera assembly 44 can be followed by a first robot arm of the robotic arm assembly 42 and then followed by a second robot arm of the robotic arm assembly 42, all of which can be inserted into the trocar 50 and hence into the internal cavity 104. Once inserted into the patient 100, the RSS 46 can move the robotic arm assembly 42 and the camera assembly 44 to an operation site manually or automatically controlled by the operator console 11.

Further disclosure regarding control of movement of individual arms of a robotic arm assembly is provided in International Patent Application Publications WO 2022/094000 A1and WO 2021/231402 A1, each of which is incorporated by reference herein in its entirety.

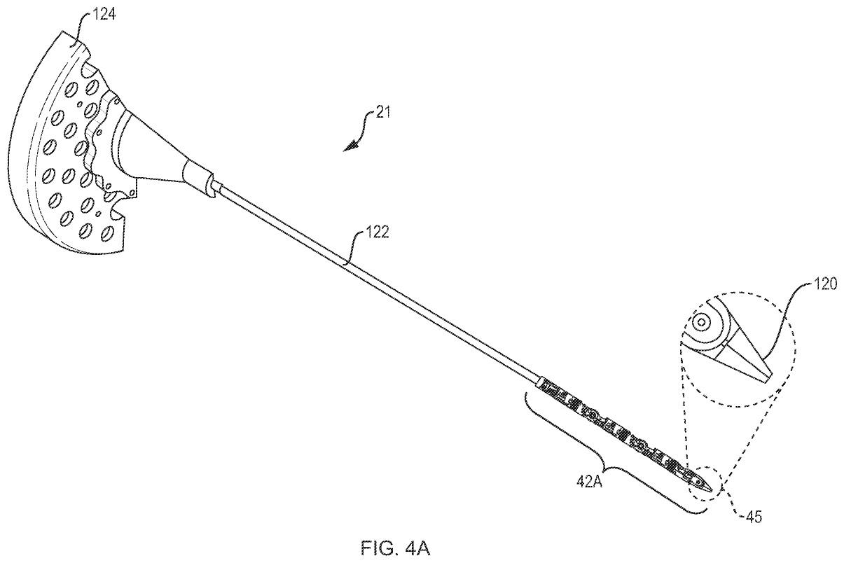

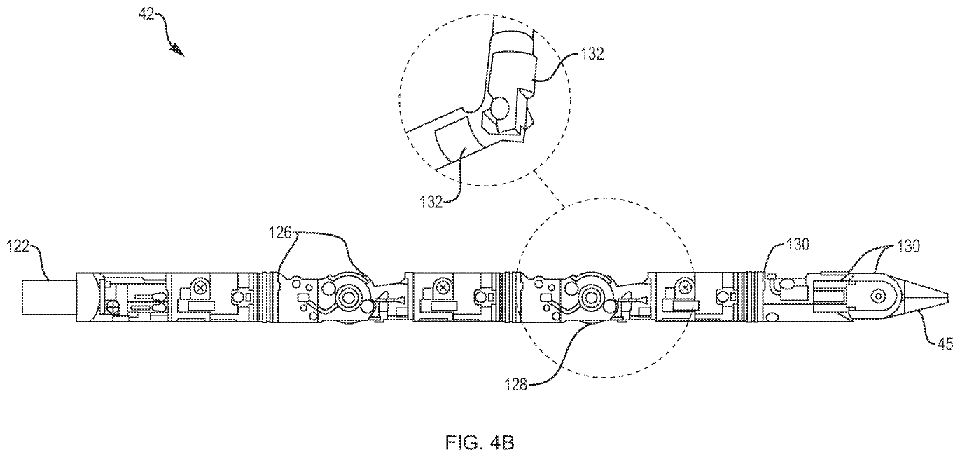

FIG. 4A is a perspective view of a robotic arm subassembly 21 in accordance with some embodiments. The robotic arm subassembly 21 includes a robotic arm 42A, the end-effector 45 having an instrument tip 120 (e.g., monopolar scissors, needle driver/holder, bipolar grasper, or any other appropriate tool), a shaft 122 supporting the robotic arm 42A. A distal end of the shaft 122 is coupled to the robotic arm 42A, and a proximal end of the shaft 122 is coupled to a housing 124 of the motor unit 40 (as shown in FIG. 2A). At least a portion of the shaft 122 can be external to the internal cavity 104 (as shown in FIGS. 3A and 3B). At least a portion of the shaft 122 can be inserted into the internal cavity 104 (as shown in FIGS. 3A and 3B).

FIG. 4B is a side view of the robotic arm assembly 42. The robotic arm assembly 42 includes a virtual shoulder 126, a virtual elbow 128 having position sensors 132 (e.g., capacitive proximity sensors), a virtual wrist 130, and the end-effector 45 in accordance with some embodiments. The virtual shoulder 126, the virtual elbow 128, the virtual wrist 130 can include a series of hinge and rotary joints to provide each arm with positionable, seven degrees of freedom, along with one additional grasping degree of freedom for the end-effector 45 in some embodiments.

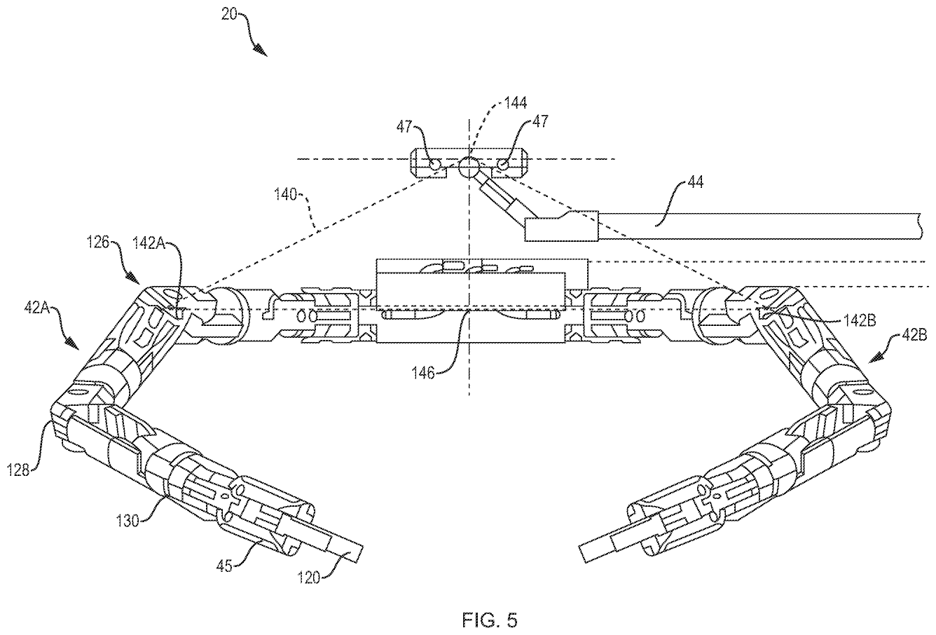

FIG. 5 illustrates a perspective front view of a portion of the robotic assembly 20 configured for insertion into an internal body cavity of a patient, which may be referred to as a surgical robotic module herein. The robotic assembly 20 includes a first robotic arm 42A and a second robotic arm 42B. The two robotic arms 42A and 42B can define, or at least partially define, a virtual chest 140 of the robotic assembly 20 in some embodiments. In some embodiments, the virtual chest 140 (depicted as a triangle with dotted lines) can be defined by a chest plane extending between a first pivot point 142A of a most proximal joint of the first robotic arm 42A (e.g., a shoulder joint 126), a second pivot point 142B of a most proximal joint of the second robotic arm 42B, and a camera imaging center point 144 of the camera(s) 47. A pivot center 146 of the virtual chest 140 lies in the middle of the virtual chest.

In some embodiments, sensors in one or both of the first robotic arm 42A and the second robotic arm 42B can be used by the system to determine a change in location in three-dimensional space of at least a portion of the robotic arm. In some embodiments, sensors in one or both of the first robotic arm and second robotic arm can be used by the system to determine a location in three-dimensional space of at least a portion of one robotic arm relative to a location in three-dimensional space of at least a portion of the other robotic arm.

In some embodiments, a camera assembly 44 is configured to obtain images from which the system can determine relative locations in three-dimensional space. For example, the camera assembly may include multiple cameras, at least two of which are laterally displaced from each other relative to an imaging axis, and the system may be configured to determine a distance to features within the internal body cavity. Further disclosure regarding a surgical robotic system including a camera assembly and associated system for determining a distance to features may be found in International Patent Application Publication No. WO 2021/159409, entitled “System and Method for Determining Depth Perception In Vivo in a Surgical Robotic System,” and published Aug. 12, 2021, which is incorporated by reference herein in its entirety. Information about the distance to features and information regarding optical properties of the cameras may be used by a system to determine relative locations in three-dimensional space.

### 2.

### 3. Trocars

Various embodiments of trocars of the present disclosure are described below with respect to FIGS. 6-22C. Some embodiments provide a trocar with independent, isolated seals that enable a surgeon to perform endoscopic surgery using multiple instruments or tools from a single, small incision. Some embodiments provide a multiple lumen trocar with independent isolated seals. In some embodiments, the trocar includes multiple seals per endoscopic instrument or tool access point for maintaining a seal of the trocar prior to, during, and after insertion of surgical material, tools, instruments, and components. In some embodiments, a configuration of the trocar enables each lumen, or at least one seal for each endoscopic tool, to translate laterally and become concentric with the cannula during insertion (e.g., translate radially toward a central insertion axis of the cannula). In some embodiments, after insertion, the portion of the endoscopic tool at the lumen and the corresponding at least one seal translate radially away from the central insertion axis of the cannula after insertion or once fully inserted.

In some embodiments, a trocar may be configured to seal around multiple, very different diameters and cross-sections (e.g., circular vs. elliptical). Typically, flexible seals (e.g., silicone seals) function best for a narrow range of diameters for which they are sized. Some surgical robotic systems include one or more robotic arms and cameras with larger instrument/tool diameters and smaller support tube/shaft diameters. In use, the support shaft or support tube would be positioned within the trocar or cannula when a wider-insertion diameter tool, instrument, robotic arm, or camera is fully inserted into an internal body cavity and in use. Alternatively, some surgical systems as taught herein include a camera assembly with a larger diameter than that of the robotic arms, and the camera assembly may be inserted into a seal with a larger diameter.

Some embodiments described herein incorporate two layers of individual seals: one layer configured to seal when nothing is inserted into the trocar or around larger diameters (e.g., for a robotic instrument arm or camera), and one to seal around smaller diameters (e.g., support tube/shaft diameters for a robotic instrument tool or camera, or laparoscopic tool shaft). The shapes and geometries of the individual seals may be any suitable geometries and need not be the same between layers or between individual seals in a same layer. In some embodiments, during insertion and extraction, an instrument may need to push the support tube/shaft of one or more of the camera or another instrument laterally out of the way in order to fit the larger “working portion” diameter of the instrument through the trocar. In some embodiments, individual seals are surrounded by one or more bellows, like accordion folds, to accommodate such a radial or lateral motion of the camera/instruments toward or away from a central insertion axis (also referred to herein as a main insertion axis) of the trocar. In some embodiments, the individual seals of one layer and the bellow(s) are all integral to one molded flexible component. In other embodiments, the bellow(s) are a separate component from the seals.

In some embodiments, a trocar may have a leakage of less than 0.3 L/min with no instruments or cameras inserted and a leakage of less than 1 L/min with all instruments or cameras inserted with an insufflation pressure in a range of 8-15 mmHg or 5-20 mmHg, and with an insufflator flow rate of less than about 20 L/min or less than about 40 L/min being used. In some embodiments, a trocar may have a leakage of between 0 L/min and 0.3 L/min with no instruments or cameras inserted and a leakage of between 0.3 L/min and 1 L/min with all instruments or cameras inserted with an insufflation pressure in a range of 8-15 mmHg or 5-20 mmHg, and with an insufflator flow rate of less than about 20 L/min or less than about 40 L/min being used. In some embodiments a trocar may have a leakage of less than 0.3 L/min with all instruments or cameras inserted with an insufflation pressure in a range of 8-15 mmHg or 5-20 mmHg, and with an insufflator flow rate of less than about 20 L/min or less than about 40 L/min being used.

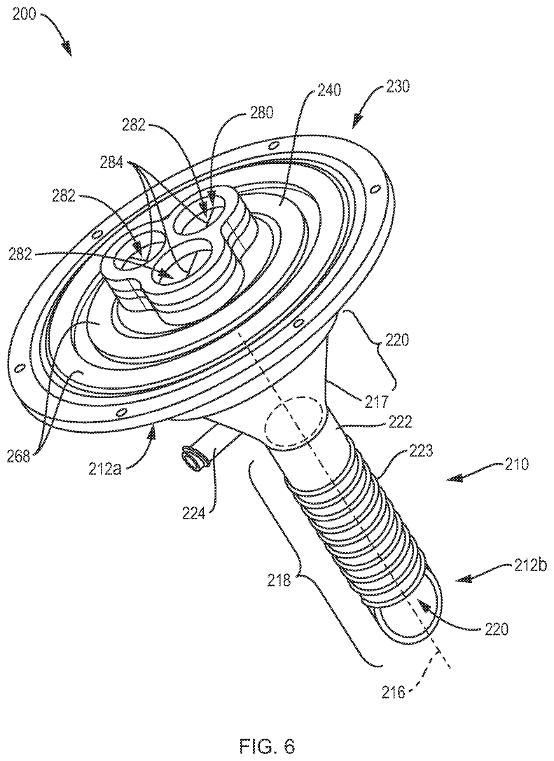

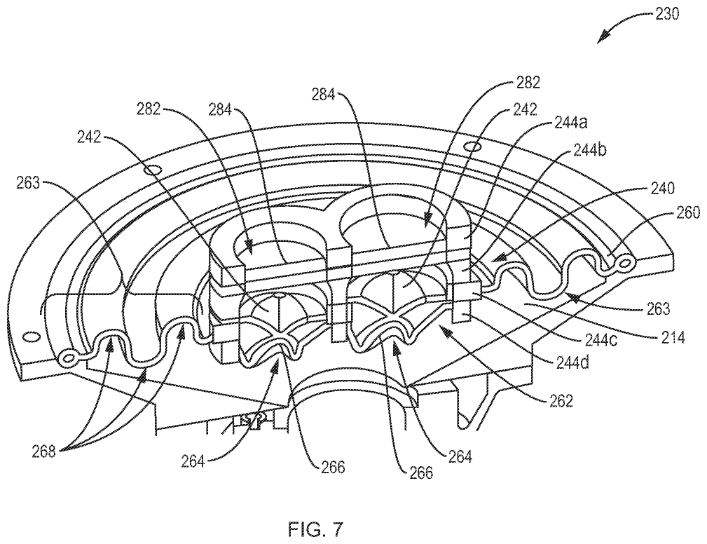

FIG. 6 provides a perspective view of a trocar 200 in accordance with some embodiments of the present disclosure. The trocar 200 includes a cannula 210 and a sealing assembly 230. The cannula 210 has a proximal end 212a, a distal end 212b, and a central insertion axis 216. The proximal end 212a defines a proximal opening 214 (depicted in FIG. 7), which is covered by the sealing assembly 230 in FIG. 6. The cannula 210 also includes central insertion axis 216, which is also the central insertion axis or the main insertion axis of the trocar. The sealing assembly 230 is configured to seal the proximal opening 214 of the cannula. In some embodiments, the cannula 210 includes a tubular portion 218 including the distal end 212b of the cannula, and includes a widened, conical or flared portion 220 including the proximal end 212a of the cannula. The tubular portion 218 defines a central lumen 222 of the cannula 210, which is the main lumen of the trocar 200. Generally, during an endoscopic procedure, the patient is prepared for surgery and an incision is formed at the surgical site. The distal end 212b of the cannula is inserted through the incision and the cannula 210 is advanced into the patient until the surgeon reaches the desired location within the patient.

Embodiments of a trocar may have various configurations and sizes to suit a variety of surgical applications and needs in accordance with some embodiments. For example, some embodiments of a trocar are suited for adult patients while others are suited for pediatric patients. Embodiments of trocar may be sized and configured for different types of procedures, such as, but not limited to, thoracic, renal, gastrointestinal, laryngeal, nasal, or brain surgeries.

To accommodate a wide number of patients and procedures, a trocar and its constituent parts may have variety of shapes and sizes in accordance with some embodiments. For example, in some embodiments, a length of the tubular portion 218 of the cannula may be in a range of about 30 mm to about 170 mm, in a range of about 60 mm to about 160 mm, or in a range of about 70 mm to about 150 mm.

In various embodiments, a cannula of a trocar may have a variety of different widths. For example, in some embodiments, an inner width (e.g., diameter) of the tubular portion 218 of the cannula, which may be referred to as a central lumen diameter or central lumen width, may be in a range of about 5 mm to about 25 mm, of about 5 mm to about 20 mm, of about 7 mm to about 20 mm, or of about 10 mm to about 19 mm. In some embodiments the central lumen diameter is about 26 mm, about 20 mm, about 19 mm, about 18 mm, about 17 mm, about 16 mm, about 15 mm, about 14 mm, about 13 mm, about 12.5 mm, about 12 mm, about 11 mm, about 10 mm, about 9 mm, about 8 mm, about 7 mm, about, 6, mm, about 5.5 mm, or about 5 mm. In some embodiments the central lumen diameter or central lumen width is less than about 26 mm, about 20 mm, about 19 mm, about 18 mm, about 17 mm, about 16 mm, about 15 mm, about 14 mm, about 13 mm, about 12 mm, about 11 mm, about 10 mm, about 9 mm, about 8 mm, about 7 mm, about 6 mm, or about 5.5 mm. Where a cannula has a circular cross-section, the inner width is the central lumen diameter. Generally, the central lumen diameter or width of the cannula 210 enables the insertion of multiple endoscopic instruments suited for the particular application and surgery type.

Cannula 210 is depicted with a circular cross section; however, in other embodiments, at least some portions of a cannula (e.g., tubular portion, widened portion, or both) may have a non-circular cross-section.

In some embodiments, an exterior surface 217 of the tubular portion 218 of a cannula may have one or more protrusions to aid in cannula retention. For example, the cannula 210 includes a cannula retention feature in the form of ribs 223 on an exterior surface 217 of the tubular portion 218 to aid in retention.

In some embodiments, the cannula 210 includes a port or connection 224 to receive gas for insufflation. For example, the cannula may include Luer lock connector or connection for insufflation tubing.

As noted above, the sealing assembly 230 is configured to seal the proximal end 212a of the cannula 210 in use. The sealing assembly 230 is configured to maintain the seal prior to, during, and after insertion of multiple endoscopic tools (e.g., one or more endoscopic instruments and an endoscopic camera) into a surgical site or internal body cavity in accordance with some embodiments. The sealing assembly 230 is also configured to maintain the seal prior to, during, and after removal of multiple endoscopic tools (e.g., one or more endoscopic instruments and an endoscopic camera) from a surgical site in accordance with some embodiments.

Referring to FIGS. 6 and 7, the sealing assembly 230 includes a seal support structure 240 that defines, at least in part, a plurality of channels 242 in accordance with some embodiments. The plurality of channels may be described as a plurality of mini-lumens herein. In some embodiments, the seal support structure 240 includes a plurality of plates 244a, 244b, 244c, 244d. In some embodiments, each of at least some or all of plates 244a-244d include a plurality of apertures, each defining a portion of a corresponding channel 242 in the plurality of channels. In some embodiments, each plate 244a-244d may have a shape corresponding to conjoined rings as shown.

The sealing assembly 230 includes a first layer 260 covering the proximal opening 214 of cannula 210. In some embodiments, the first layer 260 includes a first flexible material and has a central portion 262. In some embodiments, the first flexible material may be or include silicone. In some embodiments, the first flexible material may be or include a silicone, a rubber, or another suitable material. In some embodiments, the seal support structure 240 is attached to the central portion 262 of the first layer 260. In some embodiments, the seal support seal support structure is integral with the central portion of the first layer 260 of the sealing assembly 230.

In some embodiments, the first layer 260 of the sealing assembly 230 includes a plurality of first layer individual seals 264. Each first layer individual seal 264 has an opening 266 for insertion of an endoscopic tool or endoscopic instrument. In some embodiments, each first layer individual seal 264 corresponds to a channel 242 in the plurality of channels. In some embodiments, each first layer individual seal 264 seals an end of the corresponding channel 242.

In some embodiments, the sealing assembly 230 also includes a second layer 280 that itself includes a second flexible material and a plurality of second layer individual seals 282. Each second layer individual seal 282 has an opening 284 for insertion of an endoscopic tool or endoscopic instrument. Each first layer individual seal 264 is aligned with a corresponding second layer individual seal 282 and with a channel 242 of the plurality of channels in accordance with some embodiments. Each first layer individual seal 264 is displaced in a proximal or a distal direction from the corresponding second layer individual seal 282 (see FIG. 7) in accordance with some embodiments. For example, the trocar 200 of FIGS. 6 and 7 is configured such that each first layer individual seal 264 is displaced in a distal direction from the corresponding second layer individual seal 282. In some embodiments, each channel creates a mini-lumen for a different camera or instrument.

In some embodiments, the sealing assembly 220 is configured to enable at least part of the central portion 262 of the first layer 260 to translate radially or laterally with respect to central insertion axis 216. In some embodiments, this radial or lateral translation moves a first layer individual seal 264 and a corresponding second layer individual seal 282 closer to the central insertion axis 216 of cannula 210 in use for alignment of an endoscopic instrument or tool with central insertion axis 216 of cannula 210.

In some embodiments, the first layer 260 of the sealing assembly 230 also includes one or more concentric corrugations or pleats 268, which may also be referred to as bellows 268 herein. The bellows 268 enable radial and lateral translation of central portion 62 of first layer 260. In some embodiments, the first layer 260 also includes a peripheral portion 263. In some embodiments, the bellows 268 are disposed at the peripheral portion 263 of first layer 260. In some embodiments, the bellows 268 encircle the central portion 262 of first layer 260.

In some embodiments, at least a portion of the seal support structure (e.g., plate 244a) is disposed proximal relative to the first layer 260 and second layer 280. In some embodiments, at least a portion of the seal support structure 240 (e.g., plate 244d) is disposed distal relative to the first layer 260 and the second layer 280. In some embodiments, at least a portion of the seal support structure 240 (e.g., plate 240b) is disposed between the first layer 260 and the second layer 280.

A material or materials of the seal support structure are more rigid than the first material of the first layer and the second material of the layer in some embodiments. For example in some embodiments, the seal support structure includes one or more of high-density polyethylene (HDPE), polyoxymethylene (POM), acetal homopolymer POM (e.g., DELRIN, from Dupont), acrylonitrile butadiene styrene (ABS), polyethylene terephthalate (PET), nylon, polycarbonate, or another suitable material.

In some embodiments, each first layer individual seal 264 is or includes a universal seal. A universal seal typically includes two or more layers of overlapping leaflets. In some embodiments, each first layer individual seal includes a cross-slit valve or a duckbill valve. In some embodiments, each first layer individual seal is or includes a septum seal, a wiper seal, an hourglass or a cone seal, etc.

In some embodiments, the first layer 260 includes a seal sheet including the first flexible material, and a peripheral portion 263 of the first layer and each first layer individual seal 264 is integral with the seal sheet.

Referring again to second layer 280, in some embodiments, each second layer individual seal 282 includes a slit formed in the second flexible material. This slit forms at least a portion of the opening 284 through which an endoscopic instrument may pass during surgery. In some embodiments, each second layer individual seal is or includes a cross-slit valve or a duckbill valve. In some embodiments, each second layer individual seal is or includes a septum seal, a wiper seal, an hourglass or a cone seal, etc.

In trocar 200, the second layer individual seals 282 maintain insufflation when a smaller diameter element (e.g., a support tube/shaft) is inserted through the respective channel in accordance with some embodiments. The first layer individual seals 264 maintain insufflation when no instrument, tool, or camera is inserted through the respective channel, or each seal around a larger diameter instrument tool or camera when it is inserted through the respective channel in accordance with some embodiments.

In a trocar with multiple lumens (e.g., channels 242), each individual seal (e.g., first layer individual seal 264, second layer individual seal 282) needs to be isolated from the other seals of the same layer so that if one individual seal is compromised the others remain closed. The sealing assembly 230 of the trocar 200 isolates each of the lumens (e.g., channels 242) to create an acceptable seal if one, two, or three surgical tools are inserted into the trocar 200. In addition to isolating the individual seals (e.g., first layer individual seal 264, second layer individual seal 282), the trocar 200 employs a double layer of individual seals (e.g., first layer individual seals 264 and second layer individual seals 282) to ensure an acceptable seal prior to, during, and after insertion of surgical material, tools and/or robot components.

In some embodiments, the double layers of individual seals enable the trocar to maintain an acceptable seal prior to, during, and after insertion of tool through the trocar. Additionally, in some embodiments, the use of bellows of a flexible material (e.g., rubber or silicon sinusoidal shaped bellows), enables a seal support structure or a seal plate to radially or laterally translate to make each individual seal concentric with the cannula and incision during insertion, and then to snap back out once fully inserted.

In some embodiments, some or all of the seals may include plastic stiffener leaflets to protect them from puncture. In some embodiments, a lubricant may be employed with or included in or on at least some of the seals to ease insertion/extraction.

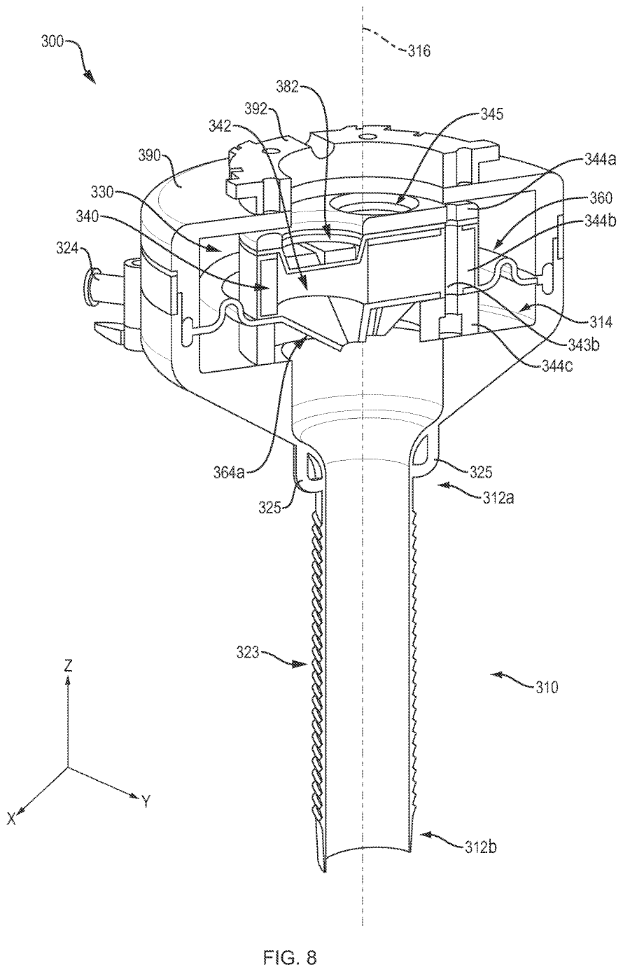

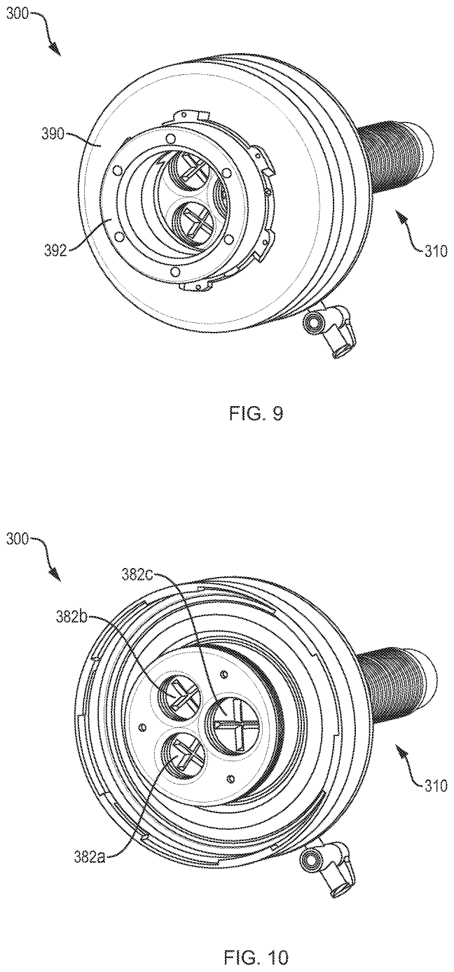

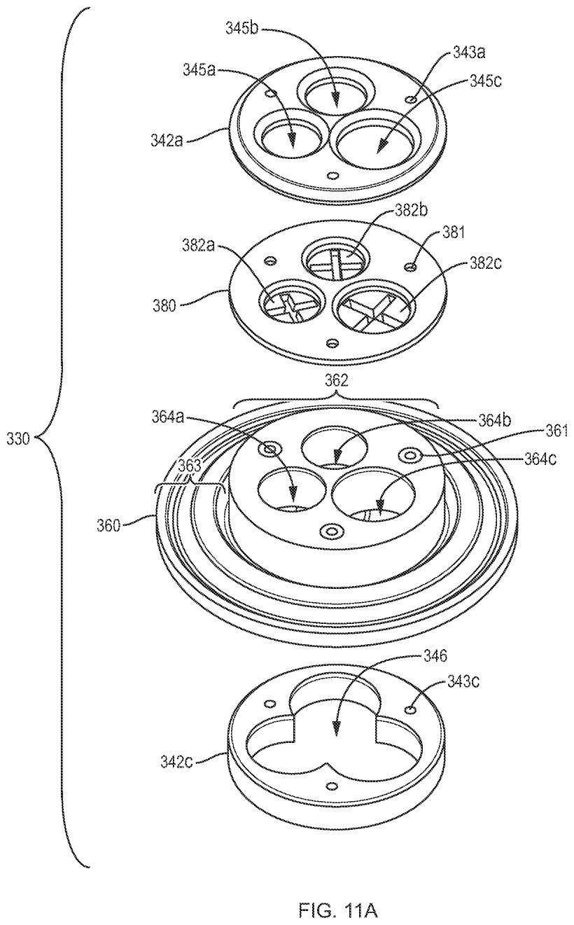

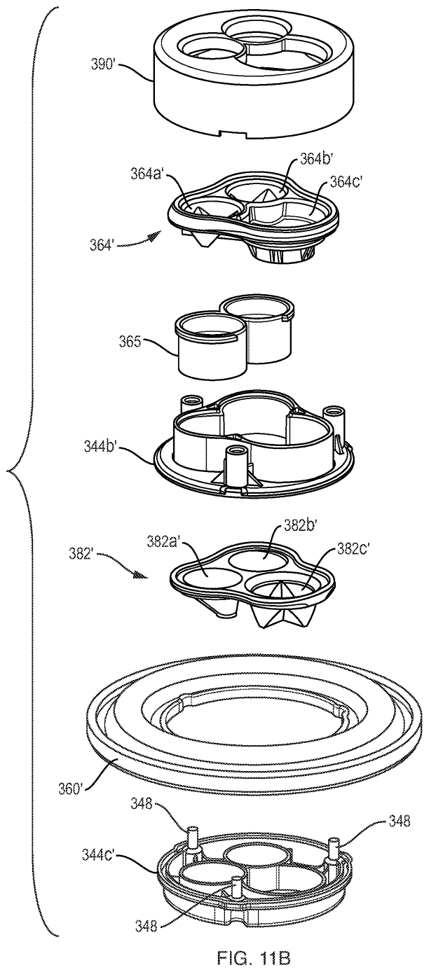

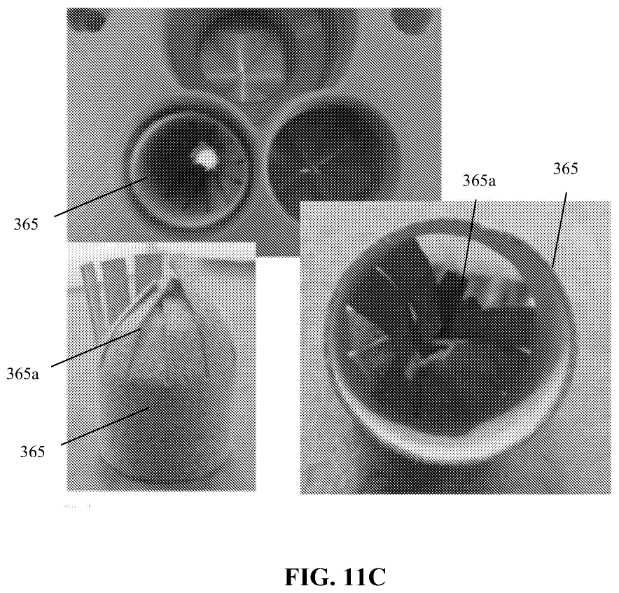

FIG. 8 depicts a cross-sectional view of another trocar 300 in accordance with some embodiments. FIG. 9 depicts a perspective view of a proximal end of the trocar 300 and FIG. 10 depicts a perspective view of a proximal end of the trocar 300 with an outer cannula cap 390 and a docking adaptor 392 removed for illustrative purposes. FIG. 11A depicts an exploded view of a sealing assembly 330 of trocar 300.

As depicted in FIGS. 8-10, the trocar 300 includes a cannula 310 having a proximal end 312a and a distal end 312b. The sealing assembly 330 seals a proximal opening 314 of the cannula. The sealing assembly 330 includes a seal support structure 340 defining, at least in part, a plurality of lumens 342. The sealing assembly 330 also includes a first layer 360 covering the proximal opening 314 of the cannula. The first layer 360 includes a first flexible material and a plurality of first layer individual seals 364 a. 364b, 364 c. In some embodiments, the first flexible material includes or is a silicone material. In some embodiments, the first layer individual seals 364a, 364b, 364c each have an asymmetric conical or funnel valve shape with a central opening as shown. In some embodiments, the first layer individual seals 364a, 364b, 364c may have some other shape. For example, seal 364a may be an hourglass seal configured to seal around a support tube of an inserted instrument, for example a camera assembly.

The first layer 360 has a central portion 362 and a peripheral portion 363 with the seal support structure 340 attached to or integral with the central portion 362 of the first layer 360. The sealing assembly 330 also includes a second layer 380 that itself includes a second flexible material and a plurality of second layer individual seals 382a, 382b, 382c. In some embodiments, each second layer individual seal 382a, 382b, 382c is a molded cross-slit seal.

Some surgical systems as taught herein include a camera assembly with a larger diameter than that of the robotic arms, and the camera assembly may be inserted into a seal with a larger diameter. For example, FIG. 10 depicts two seals 382a and 382b that are smaller in diameter than seal 382c. In such an example, the camera assembly having a larger diameter is inserted into seal 382c and the smaller diameter robotic arms are inserted into seals 382a and 382b.

In some embodiments, a peripheral portion 363 of the first layer is integral with each first layer individual seal 364a, 364b, 364c. For example, in sealing assembly 330, the first layer 360 includes a single molded piece forming the peripheral portion 363 and each first layer individual seal 364a, 364b, 364c.

In some embodiments, each second layer individual seal 382a, 382b, 382c is a portion of the same integral second layer 380.

In some embodiments, the first layer individual seals 364a, 364b, and 364c are separate from and not integral (i.e., formed as a single sheet or layer or assembly) with the first layer 360 (e.g., a bellow) and the second layer individual seals 382a, 382b, 382c are not integral (i.e., formed as a single sheet or layer or assembly) with the second layer 380. In such embodiments, a proximal seal 364′ may be situated between a cap 390′ and a plate 344b′. A distal seal 382′ may be situated between the plate 344b′ and a plate 344c′.

In embodiments where the seals are not integral with respective layers (i.e., formed as a single sheet or layer or assembly), the seals and plates may be coupled with a first layer 360′ as depicted in FIG. 11B to form a seal assembly. In some embodiments, there are three plates 344′ in the seal assembly. The distal and middle plates 344c′ and 344b′ are pressed together using pressed pins 348, which compresses the first layer 360′ and the distal seal 382′.

The middle plate 344b′ and the top plate (depicted as a component of the cap 390′) are also pressed together with pressed pins 348, which compresses the proximal seal 364′ to the first layer 360′. Lastly, the first layer 360′ is placed between a cannula body and the cap 390′, which snaps together and compresses the first layer 360′. All seals are compressed between rigid components to create an air tight seal.

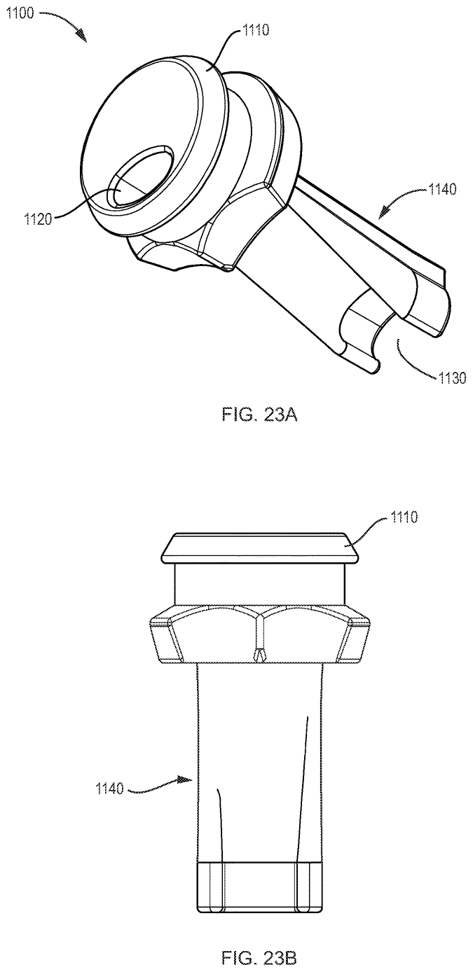

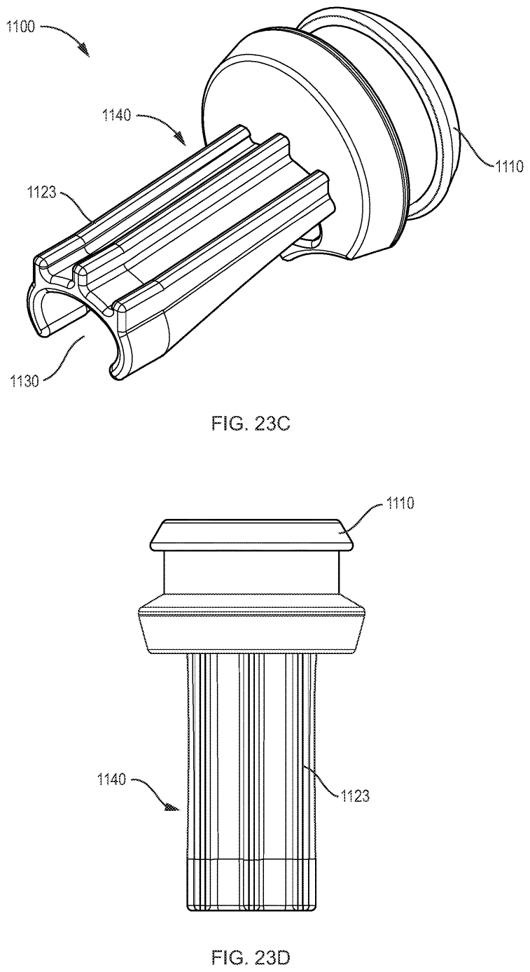

As depicted in FIG. 11B, proximal seal 364′ may include two seals 364a′ and 364b′ that can be duckbill seals (i.e., having a shape resembling a duck bill) and one seal 364c′ that may be vacant. Seals 364a′ and 364b′ may be configured to maintain a seal when no instrument or tool is inserted in the seals 364a′ and 364b'. The seal 364c′ may be configured to receive a cork or plug as described in further detail below with regards to FIGS. 23A-23D.

Distal seal 382′ may include seal 382c′ configured as a duckbill seal and two seals 382a′ and 382b′ configured as hourglass seals (i.e., a seal having a shape resembling at least a portion of an hourglass). The seal 382c′ is able to maintain a seal when no instrument or tool is inserted in the seal 382c′ and may be configured to receive a camera assembly. The seals 382a′ and 382b′ may be configured to seal around a support tube of an inserted instrument, for example a robotic arm.

In some embodiments, leaflets 365 may be added to the proximal seals 364a′ and 364b′ to help prevent the distal seals from ripping. The leaflets 365 are depicted further in FIG. 11C. A leaflet 365 may include one or more slits at a distal end of the leaflet 365 to form one or more straps 365a. The straps 365a may be configured to allow the leaflet 365 to more easily conform to the shape of a seal. In some embodiments, the leaflets 365 are formed from a polymeric material, for example polyethylene or polypropylene. Some tools have sharp edges that can cut and damage the distal seals 382b′ and 382c′. The leaflets 365 may line an inner surface of the seals 382a′ and 382b′, where rips often occur. The leaflets 365 provide a layer of plastic that prevents the sharp tools from coming into contact with the seals 382a′ and 382b′, which in turn prevents ripping. The leaflets 365 may be lubricated or inherently lubricous to add a lubricious surface that lowers the force required to insert tools into the seals 364a′ and 364b′. The leaflets 365 may be incorporated into any embodiment of the trocar discussed herein.

In some embodiments, all of the first layer individual seals have a same width or diameter. In some embodiments, at least some of the first layer individual seals have a different width or diameter than that of another of the first layer individual seals. For example, first layer individual seals 364a and 364b have a smaller diameter than that of the first layer individual seal 364c (see FIG. 11A).

In some embodiments, all of the second layer individual seals have a same width or diameter. In some embodiments, at least some of the second layer individual seals have a different width or diameter than that of another of the second layer individual seals. For example, second layer individual seals 382a and 382b have a smaller diameter than that of the second layer individual seal 382c (see FIGS. 10 and 11A).

In some embodiments, the seal support structure 340 includes multiple plates 344a, 344b, 344c. As depicted in FIGS. 8 and 11A, the seal support structure plate 342a has multiple apertures or openings 345a, 345b, 345c with each aperture or opening corresponding to a channel 342 in accordance with some embodiments. In contrast, the seal support plate 344c includes one large aperture or opening 346 through which all the channels 342 pass in accordance with some embodiments. The seal support plate 344 includes a multi-lobed opening with each lobe corresponding to a channel 342 in accordance with some embodiments.

In some embodiments, each aperture in a support plate has a same width or diameter. In some embodiments, at least some apertures in a support plate have a different width or diameter than that of another aperture. For example, apertures 345a and 345b have a smaller diameter than that of the aperture 345c.

In some embodiments, one or more of the individual seals in a layer and one or more of the channels may have a different diameter than that of another individual seal in the same layer or that of another channel. For example, one of the channels may be configured for insertion of an instrument, tool or component having a first maximum insertion diameter and another of the channels may be configured for insertion of an instrument, tool, or component having a second maximum insertion diameter larger than the first maximum insertion diameter.

The peripheral portion 363 of the first layer 360 includes a corrugation or pleat in the form of concentric bellows 368 encircling the central portion 362. The bellows 368 and the first flexible material of the first layer 360 enable the central portion 362 of the first layer, the seal support structure 340, the first layer individual seals 364a, 364b, 364c, and the second layer individual seals 382a, 382b, 382c, to shift radially or laterally with respect to the central insertion axis 316 to better align a channel 342 with the central insertion axis 316 for insertion of an instrument, tool, or camera through the channel or withdrawal of an instrument, tool or camera from the channel. In some embodiments, the trocar 300 is configured to enable each of the channels to radially or laterally shift to align with the central insertion axis 316. In some embodiments, none of the channels is aligned with the central insertion axis when no external force is exerted on the first layer.

In some embodiments, a plate 344a of the seal support structure is proximal relative to the first layer 360 and the second layer 380, a plate 344c of the seal support structure is distal relative to the first layer 360 and the second layer 380, or both. In some embodiments, a plate 344b of the seal support structure is within the first layer 360 (see FIG. 8). For example, in trocar 300, the plate 344b may be used to mold at least part of the first layer 360 and is largely encapsulated by the first layer 360.

In some embodiments, at least some of the first layer, the second layer, and components of the seal support structure are configured to be affixed to each other mechanically. For example, the first layer 360, the second layer 380, and plates 342a, 342b and 342c all include apertures or channels 361, 381, 343a, 343b, 343c, respectively, to enable them to be affixed to each other (e.g., via a screw or bolt). In some embodiments, at least some components of a trocar may be chemically joined. In some embodiments, at least some components of a trocar may be ultrasonically welded together. In some embodiments, at least some components of a trocar may be adhered to each other.

In some embodiments, the cannula 310 also includes one or more suture tie structures (e.g., suture tie loops 325) for retention and/or port site closure. In some embodiments, an external surface of the cannula 310 includes a cannula retention feature such as one or more projections (e.g., ribs 323) for retention. In some embodiments, the cannula also includes a port or connection 324 (e.g., a Luer lock connection) to receive gas for insufflation. In some embodiments, the trocar or the cannula also includes a second port or connection (e.g., a second Luer lock connection) for smoke evacuation.

In some embodiments, trocar 300 also includes outer cannula cap 390 and docking mount or docking adapter 392 attached to or integral with the outer cannula cap 392. The docking mount or docking adapter 392 enables a surgical robotic system to engage with the trocar 300. In some embodiments, a surgical robotic system may connect with an outer cannula cap, a sealing assembly or the cannula.

In some embodiments, the first layer individual seals (e.g., first layer individual seals 364a, 364b, 364c of sealing assembly 330 and the first layer individual seals 264 of sealing assembly 230) are distal relative to the second layer individual seals (e.g., the second layer individual seals 382a, 382b, 382c of sealing assembly 330 and the second layer individual seals 282 of sealing assembly 230) (see FIGS. 7 and 8) In such embodiments, the first layer individual seals 364a, 364b, 364c or 264 may be referred to as the distal seals and the second layer individual seals 382a, 382b, 382c or 282 may be referred to as the proximal seals.

In trocar 300, the first layer individual seals 364a, 364b, 364d each maintain insufflation when a smaller diameter element (e.g., a support tube/shaft) is inserted through the respective channel in accordance with some embodiments. The second layer individual seals 382a, 382b, 382c each maintain insufflation when no instrument, tool or camera is inserted through the respective channel, or each seal around a larger diameter instrument, tool, or camera when it is inserted through the respective channel in accordance with some embodiments.

In some embodiments, due to an instrument, tool, or camera, having different insertion diameters at different portions of the instrument, tool, or camera during insertion, the sealing assembly of the trocar is configured to seal around all of a range of diameters or widths for each channel. For example, in some embodiments, the sealing assembly of the trocar is configured to seal around all of a range of insertion widths including about 4 mm to about 19 mm for an instrument, tool, or camera inserted into a first channel and is configured to seal around all of a range of insertion widths including about 4 mm to about 16 mm for an instrument, tool or camera inserted in a second channel.

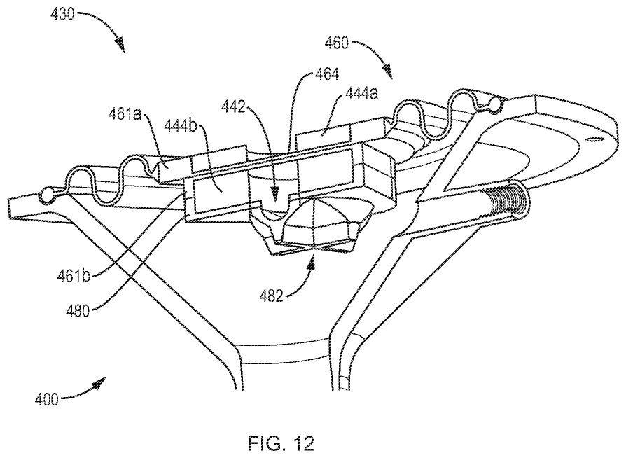

In some embodiments, a second layer of a sealing assembly may be distal relative to a first layer of the sealing assembly and second layer individual seals may be distal relative to first layer individual seals. For example, FIG. 12 depicts a trocar 400 including a sealing assembly 430 that itself includes a first layer 460 and a second layer 480, where the second layer 480 is distal relative to the first layer 460 in accordance with some embodiments. The first layer 460 includes a first flexible material and includes a plurality of first layer individual seals 464.

The second layer 480 includes a second flexible material and a plurality of second layer individual seals 482 that are distal relative to the plurality of first layer individual seals 464. In the trocar 400, the first layer individual seals 464 are the proximal seals and the second layer individual seals 482 are the distal seals. The trocar 400 also includes a seal support structure including a proximal plate 444a and a distal plate 444b that together support the first layer and second layer individual seals 464, 482, respectively. The seal support structure (e.g., the proximal plate 444a and the distal plate 444b, collectively) defines, at least in part, a plurality of channels 442, which may be referred to as a plurality of lumens or a plurality of mini-lumens herein.

In some embodiments, a first layer of a sealing assembly may include multiple sublayers. For example, the first layer 460 includes a proximal sublayer 461a and a distal sublayer 461b, with the proximal sublayer 461a and the distal sublayer 461b of the first layer 460 held between the proximal plate 444a and the distal plate 444b (see FIG. 12).

In some embodiments, at least some of the proximal sublayer 461a, the distal sublayer 461b, the second layer 480, the proximal plate 444a and the distal plate 444b may be affixed to each other using an adhesive (e.g., a glue), bonded to each other, welded to each other, or joined or affixed to each other using any other suitable method or mechanism.

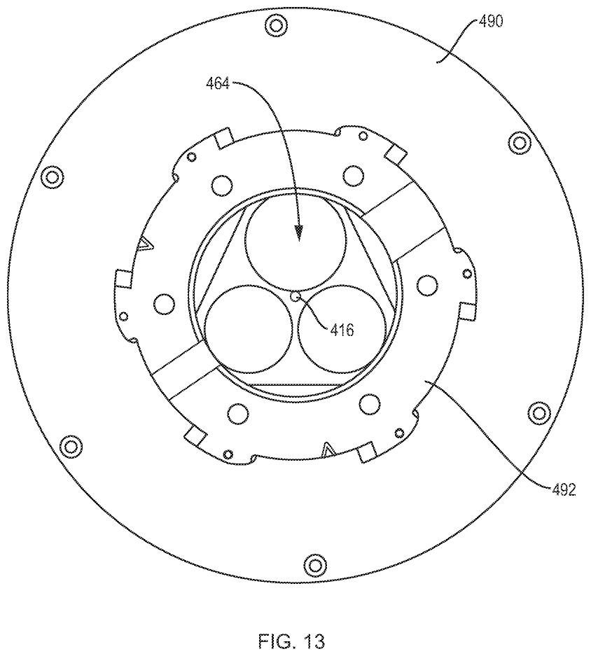

FIG. 13 is a top view of the trocar 400 with an outer cannula cap 490 and attached docking mount or adaptor 492 over a proximal end of the cannula. When no force is exerted on the first layer, all of the first layer individual seals 464 and corresponding channels are offset from a central insertion axis 464.

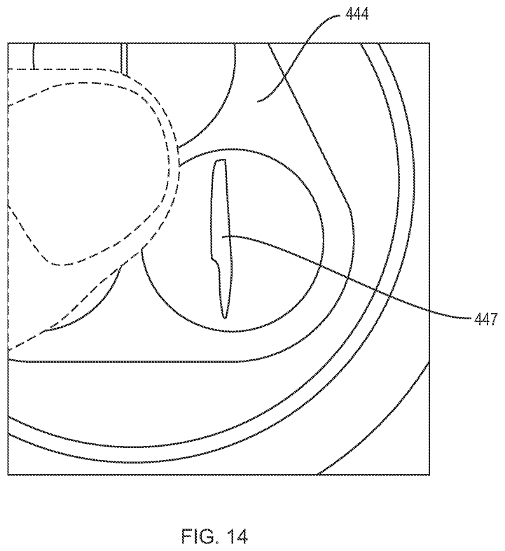

As noted above, in some embodiments, a first layer of a sealing assembly may include multiple sublayers or multiple sheets. In some embodiments, a first layer includes a first sheet including the first flexible material and a second sheet including the first flexible material. In some embodiments, each first layer individual seal includes a first slit in the first sheet and a second slit in second sheet, an orientation of the first slit being different than an orientation of the second slit forming a universal seal. In some embodiments, forming a universal seal from two components (e.g., the first sheet and the second sheet) and then cutting slits 447 in them creates an improved molding and assembly process than some other methods for forming a universal seal. FIG. 14 includes an image of individual universal seals formed from a first sheet and a second sheet of a flexible material guided by a proximal plate 444.

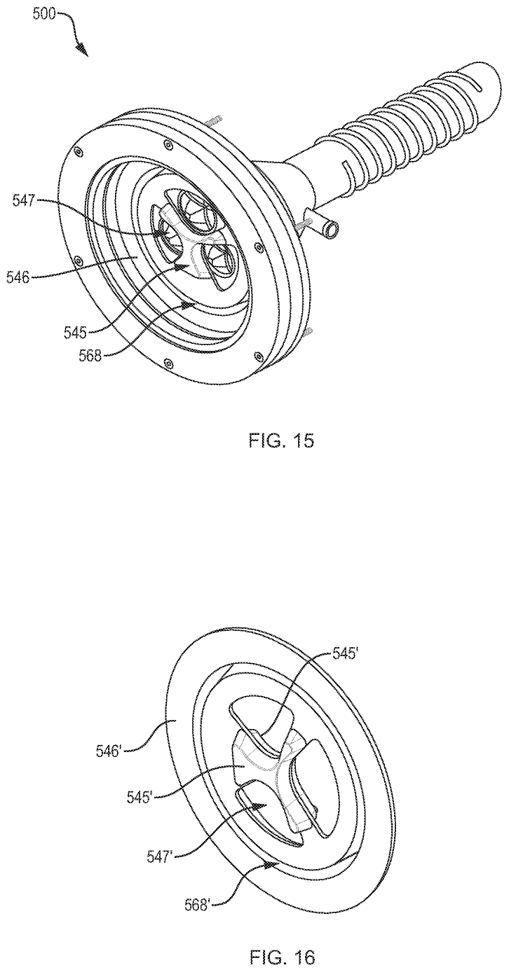

FIG. 15 depicts a trocar 500 including a brace plate 545 attached to a flexible layer 546 and FIG. 16 depicts a similar brace plate 546′ attached to a similar flexible layer 548′ where the shape of the brace plate 546′ and the shape of the flexible layer 568 in FIG. 16 are slightly different than the shape of the brace plate 546 and the shape of the flexible layer 568 in FIG. 15. The brace plate 545, 545′ is a rigid plate that includes cutouts for camera/instruments to pass through. In some embodiments, the brace plate 545, 545′ is supported on a flexible layer 546, 546′ that is in addition to the first layer and the second layer. The flexible layer 568, 568′ includes apertures 547, 547′ for camera/instruments to pass through. In some embodiments, the flexible layer 546, 546′ supporting the brace plate 545, 545′ includes bellows 568, 568′ in the form of one or more concentric corrugations or pleats encircling the brace plate 545, 545′ and the apertures 547, 547′. In some embodiments, the brace plate 545, 546′ provides mechanical support for supports (e.g., support tubes/shafts) of instruments or cameras while they experience forces by pushing them radially outwards toward a wall of a cannula lumen of the trocar. The bellows 568, 568′ enable the brace plate 545, 545′ to move radially or laterally and accommodate instrument motion toward or away from the trocar insertion axis during insertion/extraction. In FIG. 15, an outer seal layer is omitted for illustrative purposes.

A brace plate as described above may be combined with any of the embodiments above. For example, a brace plate may be layered with the individual seal layers in any order in accordance with some embodiments.

In some embodiments, a seal support structure may need more support than that provided by the first layer. In such embodiments, the second layer may also include a peripheral portion with concentric bellows and may also be used to support the seal support structure. In some embodiments, an additional flexible layer including apertures and a peripheral portion with concentric bellows may be employed to provide additional support for the seal support structure by attaching the seal support structure to the additional flexible layer.

FIG. 17 depicts a trocar 600 including a first flexible layer 660 and a rigid bellow plate 645. In FIG. 17, the second flexible layer is omitted for illustrative purposes. The first flexible layer 660 includes a plurality of first layer individual seals 664 and concentric bellows 668 encircling the first layer individual seals 664 as a group. Instead of a rigid seal support structure at least partially defining a plurality of channels, the trocar 600 includes a bellow plate 646 attached to the flexible first layer 600, where the bellow plate 646 surrounds or encircles the individual seals 664 as a group. The bellow plate 646 prevents the other seals from deforming when an instrument (e.g., a robotic arm or a camera assembly) goes through one seal and moves toward the central/main insertion axis of the trocar.

In some embodiments, a sealing assembly of a trocar includes a first layer of individual seals, peripheral bellows, and a second layer of individual seals encircled by the peripheral bellows without a rigid seal support structure (e.g., seal support plates or a bellow plate).

In some embodiments, after a first camera, instrument, or tool is inserted through the trocar and its support tube or shaft (first support tube/shaft) remains extending through the trocar, there must be sufficient room in the main cannula to insert a second first camera, instrument, or tool through the trocar even with the first support tube/shaft still in the main cannula lumen. Further, after the second camera, instrument, or tool is inserted through the trocar and its support tube or shaft (second support tube/shaft) and the first support tube/shaft remain extending through the trocar, there must be sufficient room in the main cannula lumen to insert a third camera, instrument, or tool even with the first support tube/shaft, and second support tube/shaft still in the main cannula lumen. In some embodiments, a camera, instrument, or tool employed with some trocars described herein may employ a relatively small diameter support tube/shaft to increase the available space in the main camera lumen for insertion of a subsequent camera, instrument, or tool. Reducing a diameter or width of a support tube/shaft may reduce a bending stiffness of the support tube/shaft and may increase deflection of the support tube/shaft. When a tool, instrument, or robotic arm exerts force during a procedure (e.g. to pull on tissue or suture a hernia defect closed), the less-stiff support tube/shaft may deflect from its nominal/desired position and cause unwanted effects, such as bumping into an abdominal wall or contents of the abdomen or bumping into another instrument. Some embodiments include features or components that provide mechanical support or bracing to support tubes/shafts to reduce passive deflection and force-induced deflection. Some embodiments that provide mechanical support or bracing to support tubes/shafts may enable the associated instrument to exert a greater force.

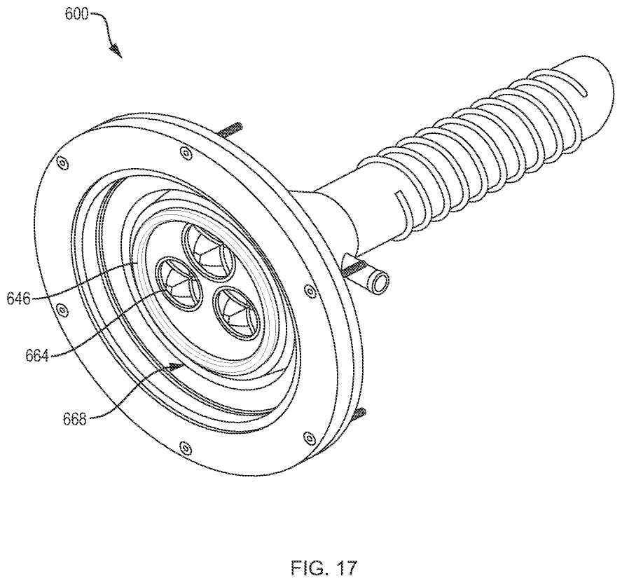

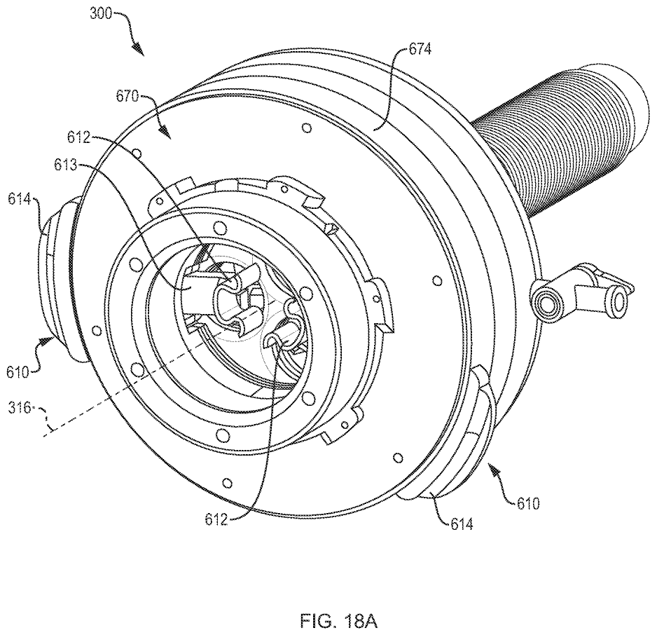

Some embodiments provide one or more sliding clips that may be disposed in a trocar cap proximal to the individual seals. FIGS. 18A-18C depict a trocar cap 670, which may also be referred to as an outer cannula cap, including sliding clips 610 attached to a trocar 300 similar to that depicted in FIGS. 9-11. In some embodiments, a sliding clip 610 may be employed for each instrument support tube/shaft. In some embodiments, sliding clips may be employed for only some of the support tubes/shafts. For example, in some embodiments sliding clips may be employed for support tubes/shafts for robotic arms, but may not be employed for a support tube /shaft for a camera assembly.

Each sliding clip 610 includes a clip portion 612 at an end of the sliding clip that extends toward a central axis 316 of the trocar 300 and of the trocar cap in accordance with some embodiments. Each sliding clip may also include an external portion, such as a finger tab portion 614, at an opposite end of the sliding clip extending away from the central axis of the trocar cap 316 and beyond a sidewall 674 of the trocar cap in accordance with some embodiments. For each sliding clip 610, the clip portion 612 is connected to the external portion (e.g., the finger tab portion 614) by a middle portion 613. In some embodiments, the middle portion 613 extends through the sidewall 674 of the trocar cap 674. Each sliding clip 612 is configured to be displaced in a radial direction toward the central axis 316 or away from the central axis by exerting force on the external portion (e.g., pulling or pushing on the finger tab portion 614).

Arrows 620 indicate displacement directions but do not correspond to a magnitude of displacement. FIG. 18C is an image of a trocar cap 674 with sliding clips 610 in which one of the sliding clips has been displaced away from the central axis.

The sliding clips 610 slide radially in and out by pulling the finger tab 614 to allow instrument and surgical material insertion. When a clip 610 is slid all the way out, it is retracted within the trocar cap 674 so that it does not obstruct the individual seal 382b, enabling an instrument (or laparoscopic tool with surgical material) to pass through the individual seal 382b.

When an instrument is fully inserted through the trocar 300, the clip portion 612 can be attached to the support tube/shaft (not shown) and the sliding clip 610 locked into place in the trocar cap 674 to brace the support tube/shaft (not shown) in the desired location. In the depicted embodiment, the locking feature includes a cantilever snap fit. In other embodiments, another locking feature or a different type of locking feature may be employed.

Example instrument insertion and extraction workflows are provided below.

To insert and brace an instrument:
1. Start position—clip is retracted into the trocar cap
2. Insert instrument through trocar
3. User slides in the clip and clips the support tube into it

To extract an instrument:
1. User removes the support tube from the clip
2. User slides the clip outward to retract it into the cap
3. Extract instrument through trocar

### 4.

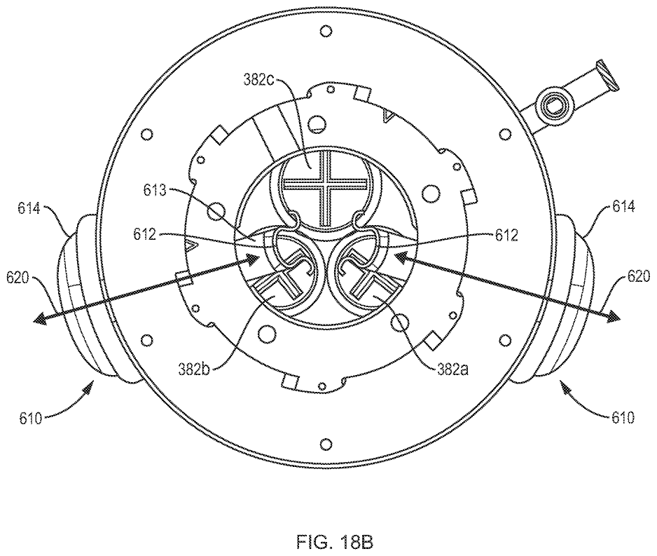

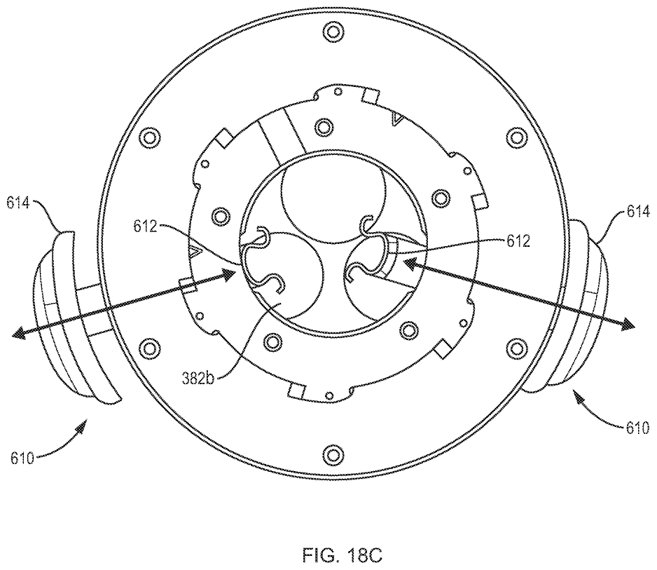

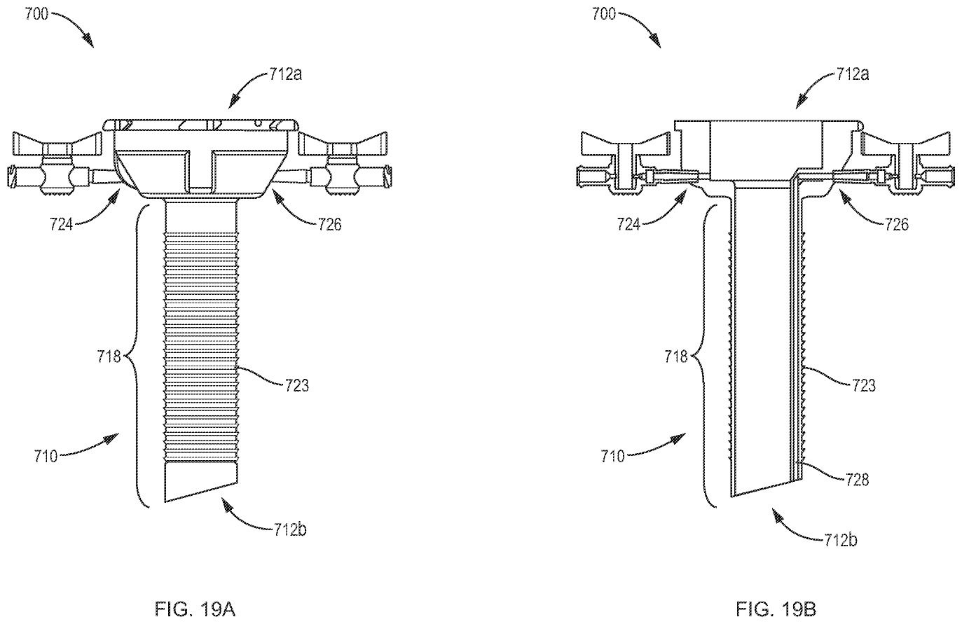

Some embodiments provide a cannula that enables smoke evacuation as well as insufflation in a single cannula. FIGS. 19A-19C depict a trocar 700 with a cannula 710 including a first port/connector to connect to a source for insufflation gas delivery, and also including a second port, connector, or connection 726 for smoke evacuation. The second port/connector 726 for smoke evacuation connects to a smoke evacuation lumen 728 of the cannula that extends to a distal tip 712b of the cannula.

The cannula 710 of trocar 700 uses a smoke evacuation lumen 728 for the smoke evacuation port/connector 726 (e.g., a Luer lock connector) separate from the cannula 710. The smoke evacuation lumen 728 extends the from the evacuation port connector 726 to the distal tip 712b of the cannula 712b. The smoke evacuation lumen 728 and second port/connector 726 enables the use of both an insufflator and a smoke evacuation machine or system with a single trocar. By having a separate lumen for smoke evacuation, the single trocar is able to connect to both the insufflator and the smoke evacuation machines and still be able to efficiently clear out smoke produced during some procedures (e.g., those employing electrocautery). Conventionally, a separate secondary trocar and associated port is employed to be connected to a smoke evacuation machine. The trocar 700 reduces a number of trocars and associated incision sites in a patent during a procedure in which insufflation and smoke evacuation is needed, which can reduce complication rates and infection risk, and can reduce procedure times by eliminating time associated with positioning a separate smoke evacuation trocar. Additionally, any trocar system that claims to be single port and supports electrocautery devices, tools or instruments would necessarily need the ability to use insufflator and smoke evacuation systems or devices with only one trocar.

The smoke evacuation lumen 728 creates a passage for air or gas to flow from an interior body cavity (e.g., an abdominal cavity) to a smoke evacuation machine enabling smoke to be removed from the interior body cavity. By extending the smoke evacuation lumen 728 to a distal tip 712 b of the cannula, an opening/inlet 729 of the smoke evacuation lumen at the distal tip 712b of the cannula draws more air/gas/smoke from the interior body cavity than it draws clean air supplied via the first insufflation port/connector 724 at a proximal portion of the cannula. The smoke evacuation lumen 728 is useful for effective smoke evacuation because if the inlet opening 729 of the smoke evacuation lumen 728 is not routed from all the way down to the cannula distal tip 712b but is instead closer to a proximal end of the cannula 712a, the inlet opening 729 would be primarily drawing clean air coming from the insufflator port/connector 724 and smoke would not be removed from the internal body cavity efficiently.

In some embodiments, the cannula 710 includes one or more cannula retention features, such as ribs 723 on an outer surface of the tubular body 718. Cannula 710 may also include any features described herein with respect to other embodiments.

The smoke evacuation port/connector 726 and smoke evacuation lumen 728 may be incorporated into any of the trocars described or disclosed herein in accordance with some embodiments. Some embodiments provide a cannula including a smoke evacuation port/connector and a smoke evacuation lumen as described herein, but do not include a sealing assembly. Some embodiments provide a trocar including a cannula that itself includes a smoke evacuation port/connector and smoke evacuation lumen as described herein, but does not include a sealing assembly. Some embodiments provide a trocar including a sealing assembly and a cannula that includes a smoke evacuation port/connector and a smoke evacuation lumen.

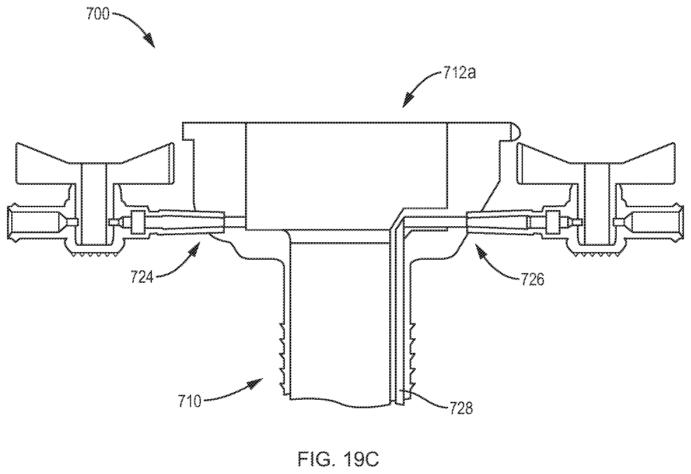

Some embodiments employ a rigid triple seal funneling trocar 800 as depicted in FIGS. 20A-20D. The trocar 800 includes a cannula having a cannula body seal 864a that is a distal seal and a trocar funnel that includes a set of individual proximal seals. In contrast to the trocars 200, 300, 400, 500, and 600, the individual proximal seals of the trocar 800 do not displace radially or laterally toward a central insertion axis of the trocar in accordance with some embodiments. The trocar 800 can accommodate three instruments/tools, for example two instruments (e.g., two robotic arms) and one camera assembly. Each instrument/tool/arm initially passes through its own separate proximal seal in the trocar funnel, then through a larger distal seal at the proximal end of the cannula portion of the trocar, the function of which is to maintain the seal when nothing is inserted).

In addition to a cannula body 810 and a trocar funnel 894, the trocar 800 may include an interface for docking the trocar 800 to a surgical robotic system. In some embodiments, the trocar funnel 894 is separable from the cannula body 810 to afford removal of material (e.g., tissue) through the main cannula lumen that would be too large to extract through one of the lumens in the trocar funnel 894.

In some embodiments, the cannula body 810 has one large lumen, also referred to as the main lumen, through which the instruments/tools (e.g., camera assembly, then both instruments) can be passed, and inside which all three support tubes/shafts sit. This main lumen has its own seal 864a at a proximal end of the cannula, which may be referred to as a distal seal because it is distal relative to the individual seals of the trocar funnel 894. The distal seal 864a of the cannula body 810 maintains insufflation when nothing is inserted into the trocar, and if the trocar funnel 894 is separated from the cannula body 810. In some embodiments, the cannula body 810 also includes a Luer lock connection for insufflation tubing that opens into the main lumen.

The trocar funnel 894 incorporates a distinct set of a lumen 842 and proximal seal 864b for each camera/instrument inserted into the trocar funnel 894. Each set of a lumen 842 and proximal seal 864b may be sized to accommodate a specific size of tool or instrument. For example, some surgical systems as taught herein include a camera assembly with a larger diameter than that of the robotic arms, and the camera assembly may be inserted into a seal with a larger diameter and the arms may be inserted into a seal with a smaller diameter. Each set of a lumen 842 and proximal seal 864b may funnel down together towards the cannula body's lumen. Sealing around each instrument/camera separately solves the problem of sealing a gap in the middle of the three seals. In the depicted embodiment, for each instrument/camera lumen, each set of a lumen 842 and proximal seal 864b follows the same trajectory towards the cannula body meaning that the seals 864b are seated at an angle. On the exterior proximal end of the trocar funnel 894, tall dividing walls function as funnels to guide the camera/instruments into their respective lumens during insertion.

The need for the trocar funnel 894 to funnel the arms into the single large lumen is that if each camera/instrument were to have its own lumen for the entire length of the cannula, the trocar diameter would be much larger than the desired size.

In some embodiments, the separate instrument/camera seals in the trocar funnel 894 may be composed of multiple layers of seals in order to maintain insufflation for both the largest diameter and the smallest diameter of the instrument/camera as that diameter varies along a length of the instrument camera. Additionally, the trocar funnel seals may incorporate plastic leaflets overtop of the seals as stiffeners.

In some embodiments, the lumen/seal trajectory in the trocar funnel 894 can be different for each set of a lumen 842 and proximal seal 864b can follow different trajectories, e.g., be oriented at an angle to one another (e.g., where the seal is not perpendicular to a central axis of the respective lumen). This includes the seals being seated “flat” or perpendicular to a central insertion axis of the cannula body 810, but not perpendicular to a central axis of the respective lumen.

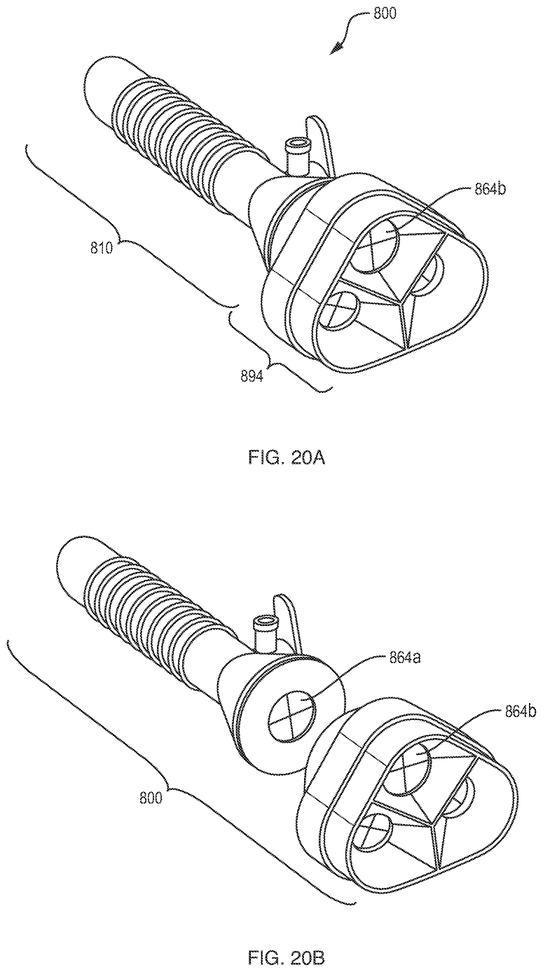

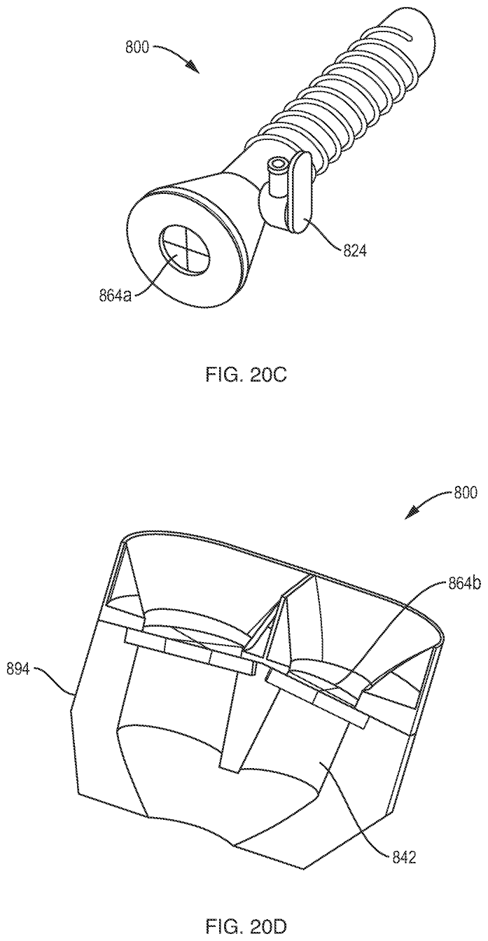

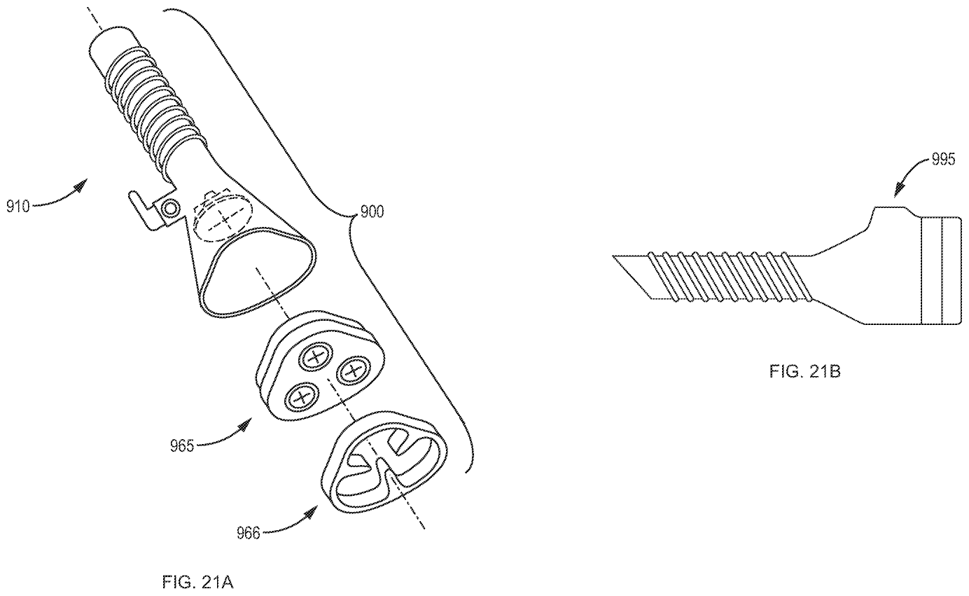

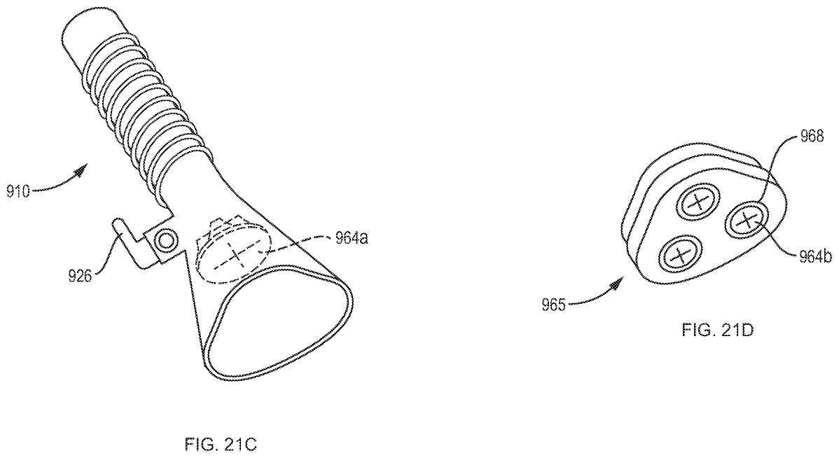

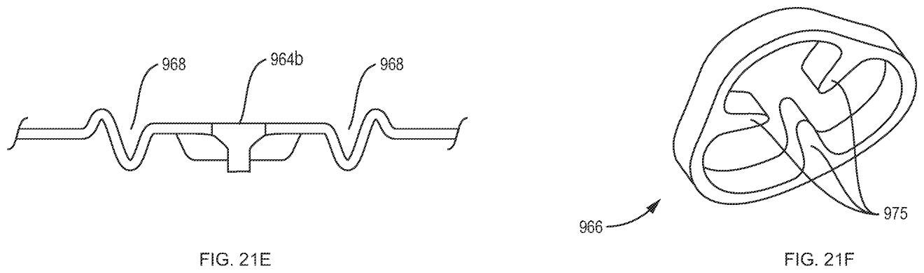

Some embodiments provide a trocar 900 with a flexible integrated seal as depicted in FIGS. 21A-21E. Trocar 900 includes a cannula body 910, a seal piece 965, and a guide gap 966. The seal piece 965 includes a triple seal including three individual seals 964b that are referred to herein as proximal seals. The cannula body 910 includes a larger central seal 964a that may referred to as distal seal. In some embodiments, the cannula body 910 also includes an interface 995 for docking the trocar with a robotic surgical system (e.g., a patient cart). The seal piece 965 and guide cap 966 form a subassembly that is separable from the cannula body 910 to enable removal of material (e.g., tissue) through the main cannula lumen and larger central seal 964a that would be too large to extract through one of the individual proximal seals 964b in the seal piece.

The cannula body 910 has one large lumen through which instruments/camera (e.g., the camera, then both instruments) can be passed, and inside which all three support tubes sit after insertion. This lumen has its own central seal 964a toward a proximal end of the lumen. The large central seal 964a maintains insufflation when nothing is inserted into the trocar, and if the seal piece-guide cap subassembly is separated from the cannula body 910. In some embodiments, the cannula body 910 includes a Luer lock connection 926 for insufflation tubing that opens into the main lumen.

In some embodiments, the seal piece 965 include a rigid plastic outer ring, over which a flexible integrated triple seal is stretched (e.g., like a silicone stretch lid). The flexible integrated triple seal includes three individual seals 964b. Sealing around each instrument/camera separately solves the problem of sealing a gap in the middle of the three.

During insertion and extraction, the instruments need to push the support tubes/shafts of the camera and other instrument out of the way in order to fit the larger “working portion” diameter of the instrument through the trocar. In order to maintain insufflation, it is beneficial for the seals to be able to radially or laterally translate and/or pivot to accommodate this. In some embodiments, the flexible integrated triple seal is composed of a single piece of flexible material (e.g., silicone) with all three seals contained in the same piece, and each individual seal is surrounded by an accordion-like “bellow” 968 that allows each seal to move and pivot independently.

The guide cap 966 features dividing walls 975, which may or may not connect in the middle, that help funnel the camera and instruments into their respective seals during insertion.

In addition to the lumen for the instrument/camera arms, the cannula body 910 may also include a smaller lumen for insufflation, a smaller lumen for smoke evacuation, or both.

In some embodiments, there may be two stacked seal pieces 965 with different integrated seals (i.e. different seal diameters/geometries) in order to maintain a seal around both the largest and smallest diameter of the arms (the diameter varies along the arm).

In some embodiments described above with respect to the trocars 200, 300, 400, 500, 600, 800, 900, any or all of the seal assembly, individual seals, or a central seal may incorporate plastic leaflets that act as stiffeners overtop of one or more of the seals.

In some embodiments, a biocompatible lubricant may be employed on one or more of the seals.

Some embodiments described above with respect to the trocars 200, 300, 400, 500, 600, 800, 900 enable a smaller nominal trocar diameter than that provided by conventional multi-lumen trocar designs that employ separate lumens for each camera/instrument along an entire length of the tubular portion of the trocar for insertion of the same diameter instruments/camera. In some embodiments, a trocar inner diameter may be less than about 25 mm, less than about 19 mm, in a range of about 13 mm to about 25 mm, in a range of about 15 to about 22 mm, or in a range of about 15 mm to 20 mm.

Unlike some trocars that employ a balloon seal, embodiments described above do not require a separate gas source from the insufflation gas source to maintain a seal.

Some embodiments include fewer components and require fewer steps in use than conventional gel-based trocars (e.g. AMT GelPOINT®).

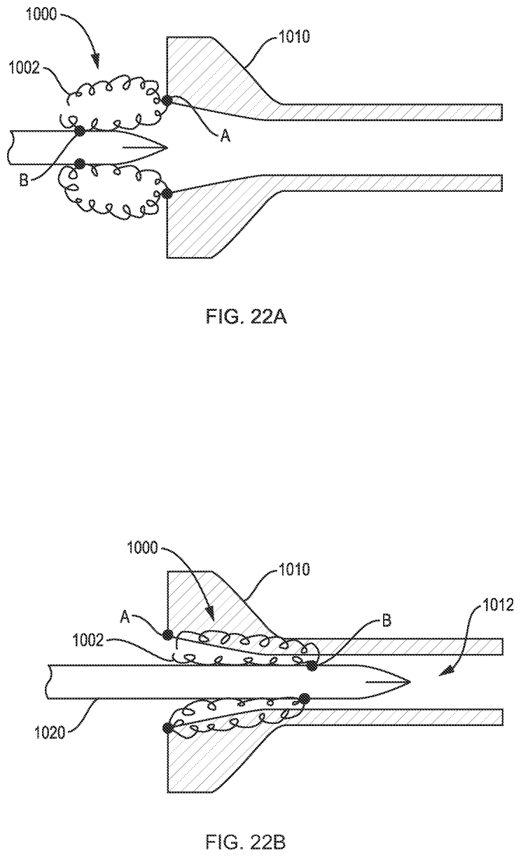

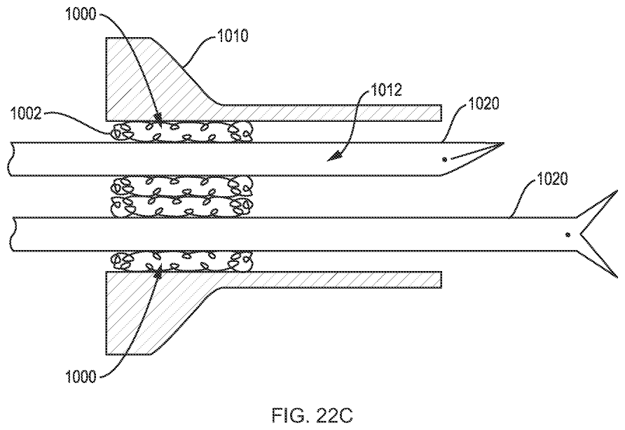

Some embodiments provide a rolling inflatable seal for single port laparoscopy as schematically depicted in FIGS. 22A and 22C. Inflatable seal 1000 prevents gas exchange between an internal body cavity (e.g., abdominal cavity) of a patient and the operating room during laparoscopic surgery. In single port surgery, where there is one incision for all the tools entering the abdomen, sealing around all of the tools is a complex challenge. In some embodiments, multiple rolling inflatable seals may be used per trocar, and each one rolling inflatable seal fits around a tool being inserted. The inflatable seal 1000 has shape of a torus elongated along a central axis and has a single continuous outer surface 1002 that has a portion forming an inward facing surface and a portion forming an outward facing surface. The portion of the outer surface forming the inward facing surface and the portion forming the outward facing surface change as the outer surface 1002 of the inflatable seal 1000 rolls. The inflatable seal 1000 forms a tight seal around an exterior of one of the laparoscopic tools 1020, and creates a uniform, compressible outer surface. For example, point A on a leading edge of the outer surface in FIG. 22A just as tool 1020 enters the lumen 1012 is rolled to a trailing edge in FIG. 22B as the tool 1020 has been advanced into the lumen 1020. Similarly, point B, which is on a trailing edge of the outer surface 1002 when tool 1020 enters the lumen in FIG. 22A is at a leading edge of the outer surface 1002 when the tool has been advanced into the lumen 1012 in FIG. 22B. When multiple rolling inflatable seals are used, each tool is independently sealed and the compression of all the seals against each and the lumen 1012 seal the internal body cavity (e.g., abdominal cavity) from the operating room (see FIG. 22C).

Some conventional inflatable air seals large enough to use for multiple tools in a single trocar require a high flow rate, a specialized trocar, and a specialized insufflator, which may be noisy. Rolling inflatable seals for a trocar may be configured so that they do not require a specialized trocar, they take up no more room around the trocar than a standard trocar, they are compatible with any number of predetermined tools, they are quiet or silent in operation, and they require no specialized tools in accordance with some embodiments.

There are conventional inflatable seals for trocars, where the seal is housed within the trocar itself, but these have the disadvantage of experiencing relative motion between the seal surface and the instruments passing through it. With irregular tool surfaces, this may lead to rips or tears, resulting in a loss of insufflation. With the inflatable rolling seal, initially there is no sliding action between the seal and the trocar or instruments. At some point during the insertion process, this will change, and the tool will slide against the rolling seal, but this can be tuned to happen in a uniform smooth area of the tool, past any tearing points. The action of rolling over sliding will also reduce the force required to insert the tool. With a seal on every tool, this also adds more redundancy as a single failure of a seal does not require complete removal of the trocar and replacement of the whole seal assembly. The rolling inflatable seal uses pressure against the instrument and the trocar interior to create a two-sided seal. The seal, a pressurized and/or compressible tube, is installed around the tip of an instrument, say a laparoscopic needle driver. The inner diameter of the seal is design to match the outer diameter of the tool (e.g., possibly bigger, the same, or smaller) and the outer diameter is sized so that it interferes with the remaining seals in such a way to prevent gas exchange.

When a tool 1020 such as a needle driver 1020 is inserted into the trocar 1010, the inflatable seal 1000 contacts the edge of the trocar 1010 and additional pressure starts to build in the inflatable seal 1000 due to compression from the trocar 1010. As the needle driver 1020 is inserted further, the distal end of the inflatable seal 1000 remains relatively stationary to the trocar (see point B), and this causes the seal 1000 to invert or roll, along the needle driver. When the needle driver 1020 is fully inserted, the inflatable seal 1000 will be fully inverted (see position of point B in FIG. 22B compared with FIG. 22A), and further distal motion along an insertion axis will be sliding motion, while extraction will be a rolling motion again.

When a second tool, say a fenestrated grasper, is prepared and inserted the same way, the seal of the fenestrated grasper will interfere with the seal of the needle driver, and pressure will increase in both seals. As the grasper is inserted, it will roll through the second seal in the same way as the needle driver did, until it is fully inserted. At that point, both seals will be within the trocar, pressing against the outer diameter of their tools, and the inner diameter of the trocar cannula. With sufficient pressure, this will prevent gas exchange between the abdomen and the operating room.

In some embodiments, the trocar may include a plug or cork 1100 as depicted in FIGS. 23A-23D. The plug or cork 1100 may be slidably connected to a support tube 1150 of a camera assembly. The plug or cork 1100 may include one or more ridges 1123 configured to provide stiffness to a stem 1140 of the cork 1100 to prevent bending during use. In some embodiments, the stem 1140 is tapered to reduce the surface area in contact with the support tube 1150 of the camera assembly to lower the force required to move the cork 1100 along the support tube 1150.

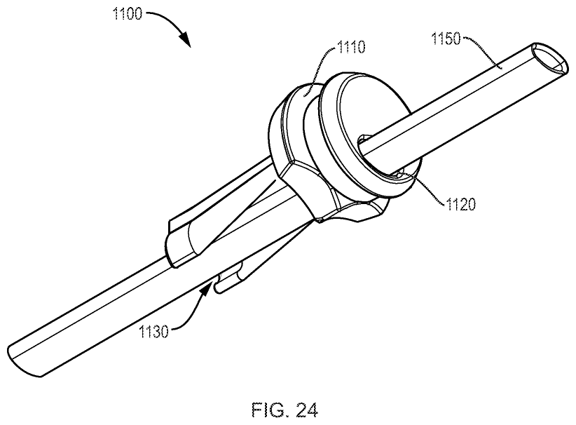

A trocar may include, for insertion of a camera assembly, a distal seal 382 that may be structured as a duckbill and a proximal seal 364 that is vacant. The cork 1100 may include an aperture 1120 through which the camera assembly support tube 1150 is seated as depicted in FIG. 24. The cork 1100 is configured to slide along the support tube 1150. For example, the support tube 1150 is seated in the aperture 1120 of the cork 1100 and extends through a support 1130 situated on an opposite end of the aperture 1120. The distal seal 382c′ may provide a complete seal when the camera assembly is absent. Once the camera assembly is inserted, the distal seal 382c′ no longer provides an adequate seal. Then a user may slide the cork 1100 on the support tube 1150 into the vacant proximal seal 364c′. The cork 1100 may include a structure 1110 that snaps into the vacant proximal seal 364c′ to form a snap fit to fill the void and adequately seal around the camera assembly.

While embodiments of the present disclosure are depicted and described herein, it will be clear to those skilled in the art that such embodiments are provided by way of example only. Numerous variations, changes, and substitutions will now occur to those skilled in the art without departing from the invention. It may be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A trocar for use in an endoscopic surgical procedure employing at least one endoscopic instrument or endoscopic camera, the trocar comprising:
a cannula having a central insertion axis and a proximal end defining a proximal opening; and
a sealing assembly configured to seal the proximal opening of the cannula, the sealing assembly including:
a seal support structure defining, at least in part, a plurality of channels;
a first layer covering the proximal opening of the cannula, the first layer having a central portion and comprising a first flexible material, the seal support structure attached to or integral with the central portion of the first layer, the first layer including a plurality of first layer individual seals each having an opening for insertion of an endoscopic tool or endoscopic instrument; and
a second layer comprising a second flexible material and including a plurality of second layer individual seals each having an opening for insertion of an endoscopic tool or endoscopic instrument, each first layer individual seal aligned with a corresponding second layer individual seal and with a channel of the plurality of channels, and displaced in a proximal or a distal direction from the corresponding second layer individual seal;
wherein the sealing assembly is configured to enable at least part of the central portion of the first layer to translate radially with respect to the central insertion axis to move a first layer individual seal and a corresponding second layer individual seal radially closer to the central insertion axis of the cannula in use for alignment of an endoscopic instrument or tool with the central insertion axis of the cannula.

2. The trocar of claim 1, wherein the first layer further comprises one or more concentric corrugations or pleats to enable radial translation of the central portion of the first layer.

3. The trocar of claim 2, wherein the first layer also has a peripheral portion, and wherein the one or more concentric corrugations or pleats are disposed in the peripheral portion of the first layer and encircle the central portion.

4. The trocar of any one of claims 1 to 3, wherein each second layer individual seal comprises a slit formed in the second flexible material.

5. The trocar of any one of claims 1 to 4, wherein each first layer individual seal comprises a universal seal.

6. The trocar of any one of claims 1 to 5, wherein each first layer individual seal comprises a cross-slit valve or a duckbill valve.

7. The trocar of any of claims 1 to 6, wherein the first layer comprises a first sheet comprising the first flexible material and a second sheet comprising the first flexible material, and wherein each first layer individual seal includes a first slit in the first sheet and a second slit in second sheet, an orientation of the first slit being different than an orientation of the second slit forming a cross-slit valve.

8. The trocar of any one of claims 1 to 7, wherein the first layer comprises a seal sheet comprising the first flexible material, and wherein a peripheral portion of the first layer and each first layer individual seal is integral with the seal sheet.

9. The trocar of claim 8, wherein the second layer comprises a second seal sheet comprising the second material, and wherein each second layer individual seal is integral with the second seal sheet.

10. The trocar of any one of claims 1 to 7, wherein the first layer individual seals are separable from the first layer and the second layer individual seals are separable from the second layer.

11. The trocar of any of claims 1 to 10, the seal support structure comprising a brace plate disposed proximal relative to the first layer and the second layer with respect to the central insertion axis; or
disposed distal relative to the first layer and the second layer with respect to the central insertion axis; or
disposed between the first layer and the second layer.

12. The trocar of claim 11, further comprising a brace plate support layer, wherein the brace plate is attached to and supported by the brace plate support layer.

13. The trocar of claim 12, wherein the brace plate support layer has central portion and a peripheral portion; and
wherein the brace plate support layer includes one or more concentric corrugations or pleats disposed in the peripheral portion and encircling the central portion.

14. The trocar of any one of claims 1 to 13, wherein the seal support structure comprises or further comprises one or more multi-lobed plates, each lobe defining an opening corresponding to an aperture in the plurality of apertures.

15. The trocar of any one of claims 1 to 13, wherein the seal support structure comprises a plurality of plates.

16. The trocar of claim 15, wherein the first layer is disposed between a first plate of the plurality of plates and a second plate of the plurality of plates.

17. The trocar of claim 16, wherein the second layer is disposed between the second of the plurality of plates and a third of the plurality of plates.

18. The trocar of claim 15, wherein the first layer and the second layer are held between the first plate of the plurality of plates and the second plate of the plurality of plates.

19. The trocar of any one of claims 1 to 13, wherein the seal support structure comprises a first plate including a plurality of apertures, each defining a portion of a corresponding channel in the plurality of channels.

20. The trocar of claim 19, wherein the seal support structure further comprises a second plate, the second plate including at least one aperture connecting with the plurality of channels.

21. The trocar of any one of claims 1 to 20, wherein the seal support structure is supported, at least in part, by the first layer.

22. The trocar of any one of claims 1 to 21, wherein the second layer covers the proximal opening of the cannula; and wherein the second layer supports or supports in part the seal support structure.

23. The trocar of claim 22, wherein the second layer has a central portion and a peripheral portion, and wherein the second layer includes one or more concentric corrugations or pleats formed in the peripheral portion surrounding the central portion.

24. The trocar of any one of claims 1 to 23, wherein the second layer is disposed proximal relative to the first layer with respect to the central insertion axis.

25. The trocar of any one of claims 1 to 23, wherein the second layer is disposed distal relative to the first layer with respect to the central insertion axis.

26. A trocar for use in an endoscopic surgical procedure employing at least one endoscopic instrument or endoscopic camera, the trocar comprising:
a cannula having a central insertion axis and a proximal end defining a proximal opening; and
a sealing assembly configured to seal the proximal end of the cannula, the sealing assembly including:
a first layer covering the proximal opening of the cannula, the first layer having a central portion and comprising a first flexible material, the first layer including a plurality of first layer individual seals in the central portion, each first layer individual seal having an opening for insertion of an endoscopic instrument or an endoscopic camera; and
a second layer comprising a second flexible material and including a plurality of second layer individual seals, each second layer individual seal having an opening for insertion of an endoscopic tool or endoscopic instrument, each first layer individual seal aligned with a corresponding second layer individual seal and displaced in a proximal or distal direction with respect to central insertion axis from the corresponding second layer individual seal;
wherein the sealing assembly is configured to enable at least part of the central portion of the first layer to translate radially with respect to the central insertion axis to move a first layer individual seal and a corresponding second layer individual seal radially closer to the central insertion axis of the cannula in use for alignment of an endoscopic instrument or tool with the central insertion axis of the cannula.

27. The trocar of claim 26, wherein the first layer, the second layer, or both define, at least in part, a plurality of lumens.

28. The trocar of claim 27 or claim 28, further comprising a seal support structure having a central aperture corresponding to the central portion of the first layer, the seal support structure affixed, either directly or indirectly, to and supported, at least in part, by the first layer.

29. The trocar of any one of claims 1 to 28, wherein for each set of a first layer individual seal and a corresponding second layer individual seal, the central portion of the first layer is configured to enable radial translation of at least part the central portion of the first layer with respect to the central insertion axis to enable movement of the first layer individual seal and the corresponding second layer individual seal radially into alignment with the central insertion axis of the cannula.

30. The trocar of any one of claims 1 to 29, wherein the sealing assembly is configured to maintain a gas and liquid seal between an ambient atmosphere and an interior of the trocar prior to and during insertion of an endoscopic instrument or endoscopic camera into the trocar in use.

31. The trocar of any one of claims 1 to 30, wherein the sealing assembly is configured to maintain a gas and liquid seal between an ambient atmosphere and an interior of the trocar during and after withdrawal of an endoscopic tool or endoscopic camera from the trocar in use.

32. The trocar of any one of claims 1 to 31, wherein each of the first layer individual seals is radially offset from the central insertion axis of the cannula when no endoscopic instruments or endoscopic cameras are inserted into the trocar.

33. The trocar of any one of claims 1 to 32, wherein the trocar is configured for use with a surgical robotic system comprising a robotic arm with an instrument and a camera assembly including a camera.

34. The trocar of claim 33, wherein the plurality of first layer individual seals includes a first layer camera seal and first layer robotic arm seal, and the plurality of second layer individual seals includes a second layer camera seal and a second layer robotic arm seal;
wherein the trocar is configured to maintain a seal when no robotic arms instruments or camera assemblies are inserted into the trocar;
wherein the trocar is configured to maintain a seal during insertion of the camera assembly into the trocar via a the first layer camera seal and the second layer camera seal; and
wherein the trocar is configured to maintain a seal during insertion of the first robotic arm into the trocar via the first layer robotic arm seal and the second layer robotic arm seal.

35. The trocar of any one of claims 1 to 34, wherein the trocar is configured for use with a surgical robotic system comprising a first robotic arm with a first instrument, a second robotic arm with a second instrument, and a camera assembly including a camera.

36. The trocar of claim 35, wherein the plurality of first layer individual seals includes a first layer camera assembly seal and a pair of first layer robotic arm seals, and the plurality of second layer individual seals includes a second layer camera assembly seal and a pair of second layer robotic arm seals;

37. The trocar of claim 36,
wherein the trocar is configured to maintain a seal when no robotic arms or camera assemblies are inserted into the trocar;
wherein the trocar is configured to maintain a seal during insertion of the camera assembly into the trocar via the first layer camera assembly seal and the corresponding second layer camera assembly seal;
wherein the trocar is configured to maintain a seal during insertion of the first robotic arm into the trocar via a first robotic arm seal of the pair of first layer robotic arm seals and a corresponding first robotic arm seal of the pair of second layer robotic arm seals; and
wherein the trocar is configured to maintain a seal during insertion of the second robotic arm into the trocar via a second robotic arm seal of the pair of first layer robotic arm seals and a corresponding second robotic arm seal of the pair of second layer robotic arm seals.

38. The trocar of claim 36 or claim 37, wherein the trocar is configured to maintain a seal when no robotic arms or camera assemblies are inserted into the trocar;
wherein the trocar is configured to maintain a seal when the camera assembly extends through the trocar via the first layer camera assembly seal and the corresponding second layer camera seal, the first robotic arm does not extend into or through the trocar, and the second robotic arm does not extend into or through the trocar;
wherein the trocar is configured to maintain a seal when the camera assembly extends through the trocar via the first layer camera assembly seal and the corresponding second layer camera seal, the first robotic arm extends through the trocar via a first robotic arm seal of the pair of first layer robotic arm seals and a corresponding first robotic arm seal of the pair of second layer robotic arm seals, and the second robotic arm does not extend into or through the trocar; and
wherein the trocar is configured to maintain a seal when the camera assembly extends through the trocar via the first layer camera assembly seal and the corresponding second layer camera seal, the first robotic arm extends through the trocar via the first robotic arm seal of the pair of first layer robotic arm seals and the corresponding first robotic arm seal of the pair of second layer robotic arm seals, and the second robotic arm extends through the trocar via a second robotic arm seal of the pair of first layer robotic arm seals and a corresponding second robotic arm seal of the pair of second layer robotic arm seals.

39. The trocar of any one of claims 36 to 38, wherein the first layer camera assembly seal and the corresponding second layer camera seal are configured for insertion of the camera assembly having a first maximum insertion diameter;
wherein a first robotic arm seal of the pair of first layer robotic arm seals and a corresponding first robotic arm seal of the pair of second layer robotic arm seals are configured for insertion of the first robotic arm having a second maximum insertion diameter smaller than the first maximum insertion diameter; and
wherein a second robotic arm seal of the pair of first layer robotic arm seals and a corresponding second robotic arm seal of the pair of second layer robotic arm seals are configured for insertion of the second robotic arm having a third maximum insertion diameter smaller than the first maximum insertion diameter.

40. The trocar of any one of claims 1 to 34, wherein a first of the first layer individual seals and a corresponding first of the second layer individual seals are configured for insertion of an endoscopic instrument or endoscopic camera having a first maximum insertion diameter;
wherein a second of the first layer individual seals and a corresponding second of the second layer individual seals are configured for insertion of an endoscopic instrument or endoscopic camera having a second maximum insertion diameter smaller than the first maximum insertion diameter; and
wherein a third of the first layer individual seals and a corresponding third of the second layer individual seals are configured for insertion of an endoscopic instrument or endoscopic camera having a third maximum insertion diameter smaller than the first maximum insertion diameter.

41. The trocar of any one of claims 1 to 40, wherein at least part of the proximal portion of the cannula includes a funnel shape or a flared shape.

42. The trocar of any one of claims 1 to 41, wherein at least some of the first layer individual seals, at least some of the second layer individual seals, or both include leaflets.

43. The trocar of claim 42, wherein the leaflets comprise a third material.

44. The trocar of claim 43, wherein the third material has a greater stiffness than that of the first material and that of the second material.

45. The trocar of any one of claims 1 to 44, wherein the seal support structure is a rigid seal support structure.

46. The trocar of any one of claims 1 to 45, wherein the proximal end of the cannula comprises at least one mating structure configured to secure the sealing assembly to the proximal end of the cannula.

47. The trocar of claim 46, wherein the sealing assembly includes at least one complimentary mating structure to secure the sealing assembly to the proximal end of the cannula.

48. The trocar from any of claims 1 to 47, wherein the cannula further comprises a retention structure on at least a portion of an exterior surface of the cannula configured to engage tissue of a patient during use.

49. The trocar from any of claims 1 to 48, wherein the cannula further comprises at least one port.

50. The trocar of claim 49, wherein the at least one port is configured to receive gas for insufflation.

51. The trocar of any one of claims 1 through 50, wherein the cannula further comprises a smoke evacuation port in a proximal portion of the cannula and a smoke evacuation lumen.

52. The trocar of claim 51, wherein the smoke evacuation lumen extends from a distal end of the cannula to the smoke evacuation port.

53. The trocar of claim 51, wherein the smoke evacuation lumen extends from a distal opening of the cannula to the smoke evacuation port.
